(19) United States
(12) Patent Application Publication
(10) Pub. No.: US 2005/0215646 A1
(43) Pub. Date: Sep. 29, 2005

(54) Methods for treating multiple sclerosis and pharmaceutical compositions therefor

(75) Inventors: Michael G. Kelly, Thousand Oaks, CA (US); Tito Serafini, San Mateo, CA (US); Hang Chen, Foster City, CA (US)

Correspondence Address: JONES DAY, 222 EAST 41ST ST, NEW YORK, NY, 10017, US

(73) Assignee: Renovis, Inc.

(21) Appl. No.: 11/076,171

(22) Filed: Mar. 8, 2005

Related U.S. Application Data
(60) Provisional application No. 60/551,882, filed on Mar. 9, 2004.
(60) Provisional application No. 60/551,902, filed on Mar. 9, 2004.

(51) Int. Cl. A61K 31/15

(52) U.S. Cl. 514/640

(57) ABSTRACT

Disclosed are pharmaceutical compositions for the treatment or prevention of chemokine mediated conditions, such as multiple sclerosis or related conditions, containing 3,4,5-trisubstituted aryl nitrones, and methods for the treatment or prevention of multiple sclerosis and related conditions. The 3,4,5-trisubstituted aryl nitrones have the formula: 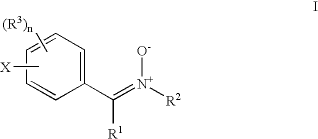
where R¹-R³ and Q are as defined in the specification, or the 3,4,5-trisubstituted aryl nitrones have the formula: 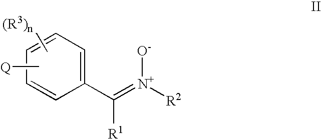
where R¹-R³ and Q are as defined in the specification.

This application claims the benefit of priority of U.S. provisional application Nos. 60/551,882 and 60/551,902, the contents of which are hereby incorporated by reference in their entireties.

### 1. FIELD OF THE INVENTION

This invention relates to the treatment and, where possible, the prevention of chemokine mediated conditions such as multiple sclerosis and related conditions in mammals including humans, and to the use of aryl nitrones for the preparation of corresponding treatment or prevention agents.

### 2. BACKGROUND OF THE INVENTION

Multiple sclerosis (MS) is an inflammatory demyelinating disorder with preservation of the axons and considered the most common cause of neurological disability in young adults. Although the mean age at onset for MS is 30 years, there are two prevalent age groups. The majority of patients are between 21 and 25 years at onset and a smaller percentage are 41 to 45 years of age. In the western world, more than 80 per 100,000 population are affected (Kurtzke, J. F. (1980) Neurology (N.Y.), 7:261-279). Several twin studies in Canada and the UK revealed that monozygotic twins are concordant on the order of 30%, compared to 2% in dizygotic twins and siblings (Ebers, G. C. et al. (1986) New Engl J Med, 315:1638-42; Mumford, C. J. et al. The British Isles Survey Of Multiple Sclerosis In Twins. (1994) Neurology, 1004:44, 11-15) and the current evidence suggests that multiple genes may interact to increase susceptibility to MS (Noseworthy (1999) Nature 399:suppl. A40-A47).

While genetics and genotyping may help to define the heritable risk for MS, their utility for diagnosis, prognosis and treatment of MS may be considerably less. It remains still unknown whether MS is a single disease and how it relates to the less common inflammatory-demyelinating CNS syndromes including neuromyelitis optica, transverse myelitis, Balo's concentric sclerosis, the Marburg variant of acute MS and acute disseminated encephalomyelitis (Noseworthy, Progress In Determining The Causes And Treatment Of Multiple Sclerosis, (1999) Nature 399:suppl. A40-A47).

Post-mortem examination of MS patients revealed the presence of multiple lesions (plaques) in the central nervous system characterized by demyelination, with relative preservation of axons, as well as gliosis and different degrees of inflammation. Although there are certain sites of predilection including the optic nerves, the spinal cord, and the periventricular regions, any part of the brain or cord can be affected (Lumsden, C. E. (1970) In Vinken P. J. Bruyn, G W, eds., Handbook of Clinical Neurology. Vol. 9. Amsterdam, North Holland, P. P. 217-309). In the majority of inflammatory neurological disorders like MS, little is known about a link between changes at a cellular and/or molecular level and nervous system structure and function.

The diagnosis remains a clinical one. Diagnosis requires the demonstration of lesions disseminated in time and space and the exclusion of other conditions that may produce the same clinical picture. Clinical classification of MS, known as the Poser criteria, includes abnormalities of evoked response and magnetic resonance images, and immunologic abnormalities in the CSF (Poser, C. M. et al. (1983) Ann. Neurol. 13: 227-231). Symptoms of MS at presentation vary among studied populations but include sensory symptoms in 24% of patients, optic neuritis in 31% of patients, limb weakness in 17% of patients and brain stem and cerebellar symptoms 25% of patients (Thompson, A. J. et al. (1986) Q. J. Med. 225:69-80). Consequently MS has a wide range of clinical presentations and courses, and the clinical course of any given patient is unpredictable. In the majority of MS patients it begins with a relapsing and remitting course, where episodes of neurological dysfunction last several weeks. Over the course of disease remissions tend be less than complete and patients pass into a progressive phase (secondary progression). During this phase of the disease patients develop severe irreversible disabilities. About one-third of patients have benign MS, which does not develop secondary progression. Approximately 10% of patients develop progressive disability from onset without relapses and remissions (primary progressive MS).

Effective therapeutic strategies remain to be identified that can reliably treat or prevent conditions such as MS. Accordingly, a need exists for novel compounds useful for treating or preventing multiple sclerosis without producing undesired side effects.

### 3. SUMMARY OF THE INVENTION

The present invention provides compositions comprising aryl nitrones that are useful for the treatment or prevention of as multiple sclerosis and related conditions.

In one aspect, the present invention provides pharmaceutical compositions for the treatment or prevention of a chemokine mediated condition such as multiple sclerosis. The pharmaceutical compositions of the invention comprise an amount of an aryl nitrone effective to treat or prevent a chemokine mediated condition such as multiple sclerosis in a pharmaceutically acceptable carrier. In a particular aspect the aryl nitrone is a 3,4,5 tri-substituted nitrone, as described herein. The compositions may be administered by a variety of routes, including, by example, orally and parenterally.

In a further aspect, the present invention provides unit dosage forms of an aryl nitrone for treating or preventing a chemokine mediated condition such as multiple sclerosis or a related condition. In certain embodiments the unit dosage forms comprise a pharmaceutical composition of an aryl nitrone in an amount effective to treat or prevent a chemokine mediated condition such as multiple sclerosis or a related condition. For instance, the unit dosage forms of the invention can comprise about 1 to 1750 mg of the aryl nitrone. Further unit dosage forms are described in detail below.

In another aspect, the present invention provides methods of treating or preventing a chemokine mediated condition such as multiple sclerosis or a related condition in a subject in need thereof. The methods comprise administering to a patient in need thereof an amount of a pharmaceutical composition comprising an aryl nitrone, such as a 3,4,5 tri-substituted nitrone, effective to treat or prevent a chemokine mediated condition such as multiple sclerosis or the related condition. By way of non-limiting example, the present pharmaceutical compositions may be administered orally or parenterally. Preferred aryl nitrones are as described herein.

In preferred embodiments of the invention, the pharmaceutical compositions of the invention comprise aryl nitrones of formula I: 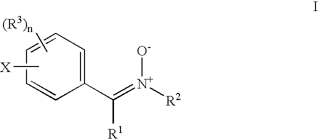
wherein
n is an integer from 1 to 4;
X is —OH or a salt thereof;
R¹ is selected from the group consisting of hydrogen, alkyl, cycloalkyl and aryl;
R² is selected from the group consisting of alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heterocycloalkyl, substituted heterocycloalkyl, heterocycloalkenyl, substituted heterocycloalkenyl, heteroaryl, substituted heteroaryl, benzyl and substituted benzyl;
each R³ is independently selected from the group consisting of aryl, heteroaryl and the following formula: 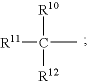
R¹⁰ is selected from the group consisting of hydrogen, lower alkyl and lower cycloalkyl; or R¹⁰ and R¹¹ can be joined to form an alkylene, substituted alkylene or heteroalkylene group;
R¹¹ and R¹² are independently selected from the group consisting of hydrogen, lower alkyl and lower cycloalkyl; or R¹¹ and R¹² can be joined to form an alkylene group having from 2 to 10 carbon atoms; and
zero, one, two or three of the carbon atoms of the phenyl ring in formula I are substituted with a heteroatom;
or a prodrug or pharmaceutically acceptable salt or solvate thereof.

In certain embodiments according to formula I, any unsaturated carbon atom of the phenyl ring is replaced with a heteroatom to yield a heteroaryl ring. For instance, any unsaturated carbon atom of the phenyl ring of formula I can be replaced with a nitrogen atom. In some embodiments, none of the unsaturated carbon atoms of the phenyl ring of formula I is replaced with a heteroatom. In other embodiments, one, two or three of the unsaturated carbon atoms of the phenyl ring of formula I are replaced with a heteroatom to yield a heteroaryl ring. In preferred embodiments, the heteroatom is a nitrogen atom. In certain aspects, the present invention provides aryl nitrone compound according to formula I, wherein one, two or three of the unsaturated carbon atoms are replaced with a heteroatom as discussed herein.

In further preferred embodiments of the invention, the pharmaceutical compositions of the invention comprise aryl nitrones of formula II: 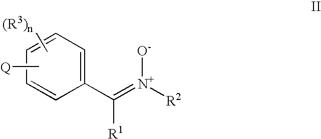
wherein
n is an integer from 1 to 4;
Q is —OR;
R is selected from the group consisting of: 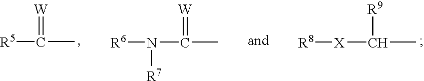
X is oxygen, sulfur, —S(O)— or —S(O)₂—; and
W is oxygen or sulfur;
R¹ is selected from the group consisting of hydrogen, alkyl, cycloalkyl and aryl;
R² is selected from the group consisting of alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heterocycloalkyl, substituted heterocycloalkyl, heterocycloalkenyl, substituted heterocycloalkenyl, heteroaryl, substituted heteroaryl, benzyl and substituted benzyl;
each R³ is independently selected from the group consisting of aryl, heteroaryl and the following formula: 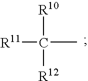
R⁵ is selected from the group consisting of alkyl, substituted alkyl, alkenyl, substituted alkenyl, alkynyl, substituted alkynyl, cycloalkyl, substituted cycloalkyl, cycloalkenyl and substituted cycloalkenyl;
R⁶ and R⁷ are independently selected from the group consisting of hydrogen, alkyl, substituted alkyl, alkenyl, substituted alkenyl, alkynyl, substituted alkynyl, cycloalkyl, substituted cycloalkyl, cycloalkenyl and substituted cycloalkenyl; or R⁶ and R⁷ can be joined to form an alkylene or substituted alkylene group having from 2 to 10 carbon atoms;
R⁸ is selected from the group consisting of alkyl, substituted alkyl, alkenyl, substituted alkenyl, alkynyl, substituted alkynyl, cycloalkyl, substituted cycloalkyl, cycloalkenyl and substituted cycloalkenyl;
R⁹ is selected from the group consisting of hydrogen, alkyl, substituted alkyl, alkenyl, substituted alkenyl, alkynyl, substituted alkynyl, cycloalkyl, substituted cycloalkyl, cycloalkenyl and substituted cycloalkenyl; or R⁸ and R⁹ can be joined to form an alkylene or substituted alkylene group having from 2 to 10 carbon atoms;
R¹⁰ is selected from the group consisting of hydrogen, lower alkyl and lower cycloalkyl; or R¹⁰ and R¹¹ can be joined to form an alkylene, substituted alkylene or heteroalkylene group;
R¹¹ and R¹² are independently selected from the group consisting of hydrogen, lower alkyl and lower cycloalkyl; or R¹¹ and R¹² can be joined to form an alkylene group having from 2 to 10 carbon atoms; and
zero, one, two or three of the carbon atoms of the phenyl ring in formula II are substituted with a heteroatom;
or a prodrug or pharmaceutically acceptable salt or solvate thereof.

In certain embodiments according to formula II, any unsaturated carbon atom of the phenyl ring is replaced with a heteroatom to yield a heteroaryl ring. For instance, any unsaturated carbon atom of the phenyl ring of formula II can be replaced with a nitrogen atom. In some embodiments, none of the unsaturated carbon atoms of the phenyl ring of formula II is replaced with a heteroatom. In other embodiments, one, two or three of the unsaturated carbon atoms of the phenyl ring of formula II are replaced with a heteroatom to yield a heteroaryl ring. In preferred embodiments, the heteroatom is a nitrogen atom. In certain aspects, the present invention provides aryl nitrone compound according to formula II, wherein one, two or three of the unsaturated carbon atoms are replaced with a heteroatom as discussed herein.

### 4. BRIEF DESCRIPTION OF THE FIGURES

FIG. 1 demonstrates the inhibition of cell chemotaxis towards CXC class chemokines by pretreatment with compound 1.

FIG. 2 demonstrates the inhibition of cell chemotaxis towards fMLP and CCR class chemokines by pretreatment with compound 1.

FIG. 3 demonstrates that compound 1 induces migration in cells expressing CXCR chemokine receptors.

FIG. 4 demonstrates that compound 1 does not induce migration in cells expressing fMLP receptors and CCR chemokine receptors.

FIG. 5 demonstrates that compounds 1-6 induce cell migration in differentiated HL-60 cells.

FIG. 6 shows experimental design for compound 1 in an in vivo mice model for multiple sclerosis.

FIG. 7 demonstrates that compound 1 is effective in the prevention and treatment of an in vivo mice model for multiple sclerosis and related conditions when administered before and during the induction of model condition. Particularly, it shows that compound 1 is effective in reducing maximum clinical scores in a chronical in vivo mice model for multiple sclerosis and related conditions. Particularly, it shows that compound 1 is effective in reducing disease severity in a chronical in vivo mice model for multiple sclerosis and related conditions.

FIG. 8 demonstrates that compound 1 is effective in the prevention and treatment of an in vivo mice model for multiple sclerosis and related conditions when administered before and during the induction of model conditions. Particularly, it shows that compound 1 is effective in reducing maximum clinical scores in a chronical in vivo mice model for multiple sclerosis and related conditions.

FIG. 9 demonstrates that compound 1 is effective in the prevention and treatment of an in vivo mice model for multiple sclerosis and related conditions when administered before and during the induction of model conditions. Particularly, it shows that compound 1 is effective in reducing cumulative disease scores in a chronical in vivo mice model for multiple sclerosis and related conditions.

FIG. 10 demonstrates that compound 1 is effective in the prevention and treatment of an in vivo mice model for multiple sclerosis and related conditions when administered before and during the induction of model conditions. Particularly, it shows that compound 1 is effective in reversing disease-related weigh loss in a chronical in vivo mice model for multiple sclerosis and related conditions.

FIG. 11 demonstrates that compound 1 is effective in the prevention and treatment of an in vivo mice model for multiple sclerosis and related conditions when administered before and during the induction of model conditions. Particularly, it shows that compound 1 is effective in reducing disease-associated inflammatory infiltration in a chronical in vivo mice model for multiple sclerosis and related conditions.

FIG. 12 demonstrates that compound 15 is effective in the prevention of an in vivo rat model for multiple sclerosis and related conditions.

FIG. 13 demonstrates that compound 15 is effective in the treatment of an in vivo rat model for multiple sclerosis and related conditions when administered after induction of the model condition.

FIG. 14 demonstrates that compound 15 is effective in the treatment of an in vivo rat model for multiple sclerosis and related conditions when administered at the peak of the model condition.

### 5. DETAILED DESCRIPTION OF THE INVENTION

The present invention is based, in part, on the discovery that aryl nitrones of the invention are useful for the treatment or prevention of multiple sclerosis and related disorders in subjects in need thereof. Accordingly, the present invention provides compositions comprising the aryl nitrones and methods of their use for treating or preventing multiple sclerosis or related disorders.

5.1 Definitions

When describing the aryl nitrones, pharmaceutical compositions and methods of this invention, the following terms have the following meanings unless otherwise specified.

“Acyl” refers to the group —C(O)R where R is hydrogen, alkyl, aryl or cycloalkyl.

“Acylamino” refers to the group —NRC(O)R where each R is independently hydrogen, alkyl, aryl or cycloalkyl.

“Acyloxy” refers to the group —OC(O)R where R is hydrogen, alkyl, aryl or cycloalkyl.

“Alkenyl” refers to a monovalent branched or unbranched unsaturated hydrocarbon group preferably having from 2 to 10 carbon atoms and more preferably 2 to 8 carbon atoms and having at least 1 and preferably from 1-2 sites of carbon-carbon double bond unsaturation. Preferred alkenyl groups include ethenyl (—CH═CH₂), n-propenyl (—CH₂CH═CH₂), isopropenyl (—C(CH₃)═CH₂), and the like.

“Substituted alkenyl” refers to an alkenyl group having 1 or more substituents, for instance from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkoxy” refers to the group —OR where R is alkyl. Preferred alkoxy groups include, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

“Substituted alkoxy” refers to an alkoxy group having 1 or more substituents, for instance from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkoxycarbonyl” refers to the group —C(O)OR where R is alkyl or cycloalkyl.

“Alkoxycarbonylamino” refers to the group —NRC(O)OR′ where R is hydrogen, alkyl, aryl or cycloalkyl, and R′ is alkyl or cycloalkyl.

“Alkyl” refers to a monovalent branched or unbranched saturated hydrocarbon group preferably having from 1 to about 11 carbon atoms, more preferably from 1 to 8 carbon atoms and still more preferably 1 to 6 carbon atoms. This term is exemplified by groups such as methyl, ethyl, n-propyl, isopropyl, n-butyl, isobutyl, tert-butyl, n-hexyl, n-octyl, tert-octyl and the like. The term “lower alkyl” refers to an alkyl group having from 1 to 11 carbon atoms.

“Substituted alkyl” refers to an alkyl group having 1 or more substituents, for instance from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkylene” refers to a divalent branched or unbranched saturated hydrocarbon group preferably having from 1 to 10 carbon atoms and more preferably from 1 to 6 carbon atoms. This term is exemplified by groups such as methylene (—CH₂—), ethylene (—CH₂CH₂—), the propylene isomers (e.g., CH₂CH₂CH₂—and —CH(CH₃)CH₂—) and the like.

“Substituted alkylene” refers to an alkylene group having 1 or more substituents, for instance from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkynyl” refers to a monovalent branched or unbranched unsaturated hydrocarbon group preferably having from 2 to 10 carbon atoms and more preferably 2 to 6 carbon atoms and having at least 1 and preferably from 1-2 sites of carbon-carbon triple bond unsaturation. Preferred alkynyl groups include ethynyl (—C═CH), propargyl (—CH₂C═CH) and the like.

“Substituted alkynyl” refers to an alkynyl group having 1 or more substituents, for instance from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Amino” refers to the group —NH₂.

“Substituted amino” refers to the group —N(R)₂ where each R is independently selected from the group consisting of hydrogen, alkyl, substituted alkyl, alkenyl, substituted alkenyl, alkynyl, substituted alkynyl, aryl, cycloalkyl, substituted cycloalkyl, and where both R groups are joined to form an alkylene group. When both R groups are hydrogen, —N(R)₂ is an amino group.

“Aminocarbonyl” refers to the group —C(O)NRR where each R is independently hydrogen, alkyl, aryl and cycloalkyl, or where the R groups are joined to form an alkylene group.

“Aminocarbonylamino” refers to the group —NRC(O)NRR where each R is independently hydrogen, alkyl, aryl or cycloalkyl, or where two R groups are joined to form an alkylene group.

“Aminocarbonyloxy” refers to the group —OC(O)NRR where each R is independently hydrogen, alkyl, aryl or cycloalkyl, or where the R groups are joined to form an alkylene group.

“Aryl” refers to an unsaturated aromatic carbocyclic group of from 6 to 14 carbon atoms having a single ring (e.g., phenyl) or multiple condensed rings (e.g., naphthyl or anthryl). Preferred aryls include phenyl, biphenyl, naphthyl and the like. Unless otherwise constrained by the definition for the individual substituents, such aryl groups can optionally be substituted with 1 or more substituents, for instance from 1 to 5 substituents, preferably 1 to 3 substituents, selected from the group consisting of acyl, acylamino, acyloxy, alkenyl, substituted alkenyl, alkoxy, substituted alkoxy, alkoxycarbonyl, alkyl, substituted alkyl, alkynyl, substituted alkynyl, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, nitro, thioalkoxy, substituted thioalkoxy, thioaryloxy, thiol, alkyl-S(O)—, aryl-S(O)—, alkyl-S(O)₂— and aryl-S(O)₂—.

“Aryloxy” refers to the group —OR where R is aryl.

“Cycloalkyl” refers to a cyclic alkyl group of from 3 to 10 carbon atoms having a single cyclic ring or multiple condensed or bridged rings which can be optionally substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, or multiple or bridged ring structures such as adamantanyl and the like. The term “lower cycloalkyl” refers to a cycloalkyl group having from 3 to 6 carbon atoms.

“Substituted cycloalkyl” refers to a cycloalkyl group having 1 or more substituents, for instance from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Cycloalkoxy” refers to the group —OR where R is cycloalkyl. Such cycloalkoxy groups include, by way of example, cyclopentoxy, cyclohexoxy and the like.

“Cycloalkenyl” refers to a cyclic alkenyl group of from 4 to 10 carbon atoms having a single cyclic ring and at least one point of internal unsaturation which can be optionally substituted with from 1 to 3 alkyl groups. Examples of suitable cycloalkenyl groups include, for instance, cyclopent-3-enyl, cyclohex-2-enyl, cyclooct-3-enyl and the like.

“Substituted cycloalkenyl” refers to a cycloalkenyl group having 1 or more substituents, for instance from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

As used herein, the term “cycloheteroalkyl” refers to a stable heterocyclic non-aromatic ring and fused rings containing one or more heteroatoms independently selected from N, O and S. A fused heterocyclic ring system may include carbocyclic rings and need only include one heterocyclic ring. Examples of heterocyclic rings include, but are not limited to, piperazinyl, homopiperazinyl, piperidinyl and morpholinyl, and are shown in the following illustrative examples: 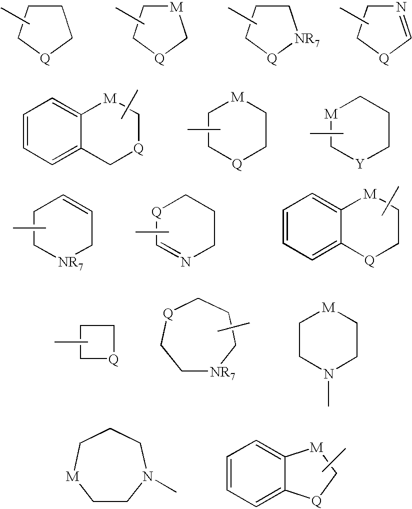
optionally substituted with one or more groups selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—. Substituting groups include carbonyl or thiocarbonyl which provide, for example, lactam and urea derivatives. In the examples, M is CR⁷, NR₂, O, or S; Q is O, NR₂ or S. R⁷ and R⁸ are independently selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

As used herein, the term “heteroaryl” refers to an aryl ring system having one to four heteroatoms as ring atoms in a heteroaromatic ring system, wherein the remainder of the atoms are carbon atoms. Suitable heteroatoms include oxygen, sulfur and nitrogen. Preferably, the heterocyclic ring system is monocyclic or bicyclic. Nonlimiting examples include the following, which may be substituted with one or more R⁷: 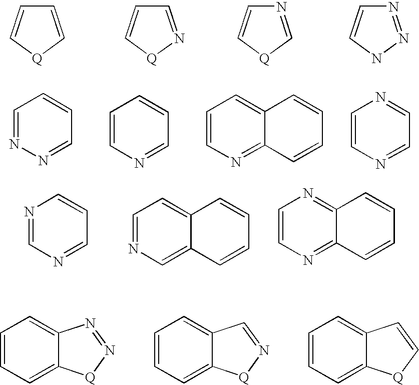
wherein R⁷ and R⁸ are each independently selected from hydrogen, lower alkyl, alkyl, alkenyl, alkynyl, cycloheteroalkyl, alkanoyl, alkoxy, aryloxy, heteroaryloxy, alkylamino, arylamino, heteroarylamino, NR¹¹COR¹², NR¹¹SOₘR¹² where m=1 or 2, COOalkyl, COOaryl, CONR¹¹R¹², CONR¹¹R¹², NR¹¹R¹², SO₂NR¹¹R¹², S(O)n-alkyl or S(O)n-aryl where n is 0, 1 or 2; R⁷ and R⁸ may be joined to form a cyclic ring (saturated or unsaturated) from 5 to 8 atoms, optionally containing one or more heteroatoms selected from the group N, O or S; and R¹¹, R¹², and R¹² are independently hydrogen, alkyl, alkenyl, alkynyl, perfluoroalkyl, cycloalkyl, cycloheteroalkyl, aryl or heteroaryl;

“Halo” or “halogen” refers to fluoro, chloro, bromo and iodo. Preferred halo groups are either fluoro or chloro.

“Hydroxyl” refers to the group —OH.

“Keto” or “oxo” refers to the group ═O.

“Nitro” refers to the group —NO₂.

“Thioalkoxy” refers to the group —SR where R is alkyl.

“Substituted thioalkoxy” refers to a thioalkoxy group having 1 or more substituents, for instance from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Sulfone” refers to the group —SO₂R. In preferred embodiments, R is selected from H, lower alkyl, alkyl, aryl and heteroaryl.

“Thioaryloxy” refers to the group —SR where R is aryl.

“Thioketo” refers to the group ═S.

“Thiol” refers to the group —SH.

The term “subject” refers to an animal such as a mammal, including, but not limited to, primate (e.g., human), cow, sheep, goat, horse, dog, cat, rabbit, rat, mouse and the like. In preferred embodiments, the subject is a human.

The terms “treat,” “treating” or “treatment,” as used herein, refer to a method of alleviating or abrogating a disorder and/or its attendant symptoms. The terms “prevent,” “preventing” or “prevention,” as used herein, refer to a method of barring a subject from acquiring a disorder and/or its attendant symptoms. In certain embodiments, the terms “prevent,” “preventing,” or “prevention,” refer to a method of reducing the risk of acquiring a disorder and/or its attendant symptoms.

“Pharmaceutically acceptable salt” refers to any salt of a compound of this invention which retains its biological properties and which is not biologically or otherwise undesirable. Such salts may be derived from a variety of organic and inorganic counter-ions well known in the art and include, by way of example illustration, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like. The term “pharmaceutically- acceptable cation” refers to a pharmaceutically acceptable cationic counter-ion of an acidic functional group. Such cations are exemplified by sodium, potassium, calcium, magnesium, ammonium, tetraalkylammonium cations, and the like.

“Prodrugs” refers to compounds which have metabolically cleavable groups that can become by solvolysis or under physiological conditions the compounds of the invention which are pharmaceutically active in vivo. Such examples include, but are not limited to, ethers, thioethers, esters, thioesters, choline esters and the like, N-alkylmorpholine esters and the like.

“Solvate” refers to a compound of the present invention or a salt thereof, that further includes a stoichiometric or non-stoichiometric amount of solvent bound by non-covalent intermolecular forces. Where the solvent is water, the solvate is a hydrate.

The term “label” refers to a display of written, printed or graphic matter upon the immediate container of an article, for example the written material displayed on a vial containing a pharmaceutically active agent.

The term “labeling” refers to all labels and other written, printed or graphic matter upon any article or any of its containers or wrappers or accompanying such article, for example, a package insert or instructional videotapes or DVDs accompanying or associated with a container of a pharmaceutically active agent.

In some cases, the aryl nitrones of this invention may contain one or more chiral centers. Typically, such compounds will be prepared as a racemic mixture. If desired, however, such compounds can be prepared or isolated as pure stereoisomers, i.e., as individual enantiomers or diastereomers, or as stereoisomer-enriched mixtures. All such stereoisomers (and enriched mixtures) of the aryl nitrones of formula I are included within the scope of this invention. Pure stereoisomers (or enriched mixtures) may be prepared using, for example, optically active starting materials or stereoselective reagents well known in the art. Alternatively, racemic mixtures of such compounds can be separated using, for example, chiral column chromatography, chiral resolving agents and the like.

Additionally, all geometric isomers of the nitrone compounds of formula I or formula II are included within the scope of this invention including, for example, all isomers (i.e. E and Z isomers) of the carbon-nitrogen double bond of the nitrone functionality.

As used herein, the term “about” refers to a range of tolerance above or below a quantitative amount known to be acceptable to those of skill in the art. For instance, a dose of about 1000 mg indicates a dose typically administered under the guidance of a practitioner when a dose of 1000 mg is indicated. In certain embodiments, the term “about” refers to ±10% or ±5%.

### 5.2 Methods and Compositions for Treating or Preventing Chemokine Mediated Conditions Such as Multiple Sclerosis And Related Conditions

The present invention provides compositions comprising aryl nitrones of the invention and methods of their use for treating or preventing a condition associated with chemokine function in a subject. In preferred embodiments, the aryl nitrones are according to Formula I. Preferred methods of treatment or prevention include methods of treating or preventing chemokine mediated conditions such as multiple sclerosis or related conditions. Related conditions include demyelinating disorders and also include, for instance, central pontine myelinolysis, Guillain-Barre syndrome and leukodystrophy.

In the methods of treatment, the subject can be any mammalian subject presenting one or more symptoms associated with a condition associated with chemokine function, multiple sclerosis or a related condition according to the judgment of one of skill in the art. In the methods of prevention, the subject can be any mammalian subject at risk for such a condition. In particularly preferred embodiments, the subject is a primate or a human.

In the further method of treatment or prophylaxis aspects, this invention provides a method of treating or prophylaxing a mammal susceptible to or afflicted with a condition related to chemokine function such as a neurodegenerative disease, a peripheral neuropathy, an infection, a sequela of an infection, or an autoimmune disease, which method comprises administering an effective amount of one or more of the pharmaceutical compositions just described.

In certain embodiments of the invention, the methods and compositions can be administered so as inhibit chemokine activity or function for the treatment of diseases that are associated with inflammation, including but not limited to, inflammatory or allergic diseases such as asthma, allergic rhinitis, hypersensitivity lung diseases, hypersensitivity pneumonitis, eosinophilic pneumonias, delayed-type hypersensitivity, interstitial lung disease (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies, insect sting allergies; autoimmune diseases, such as rheumatoid arthritis, psoriatic arthritis, systemic lupus erythematosus, myastenia gravis, juvenile onset diabetes; glomerulonephritis, autoimmune throiditis, Alopecia Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Cicatricial Pemphigoid, CREST Syndrome, Cold Agglutinin Disease, Crohn's Disease, Discoid Lupus, Essential Mixed Cryoglobulinemia, Fibromyalgia-Fibromyositis, Graves' Disease, Guillain-Barré, Hashimoto's Thyroiditis, Idiopathic Pulmonary Fibrosis, Idiopathic Thrombocytopenia Purpura, IgA Nephropathy, Insulin-dependent Diabetes, Juvenile Arthritis, Lichen Planus, Lupus, Ménière's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjögren's Syndrome, Stiff-Man Syndrome, Takayasu Arteritis, Temporal Arteritis/Giant Cell Arteritis, Ulcerative Colitis, Uveitis, Vasculitis, Vitiligo, Wegener's Granulomatosis, Churg-Strauss Syndrome, Atopic Allergy, Autoimmune Atrophic Gastritis, Achlorhydra Autoimmune, Cushings Syndrome, Dermatomyositis, Erythematosis, Goodpasture's Syndrome, Idiopathic Adrenal Atrophy, Lambert-Eaton Syndrome, Lupoid Hepatitis, Lymphopenia, Phacogenic Uveitis, Primary Sclerosing Cholangitis, Schmidt's Syndrome, Sympathetic Ophthalmia, Systemic Lupus Erythematosis, Thyrotoxicosis, Type B Insulin Resistance, Autoimmune ureitis, Autoimmune oophoritis and orchitis, Dermatitis herpetiformis.graft rejection, including allograft rejection or graft-versus-host disease; inflammatory bowel diseases, such as Crohn's disease and ulcerative colitis; spondyloarthropathies; scleroderma; psoriasis (including T-cell mediated psoriasis) and inflammatory dermatoses such as dermatitis, eczema, atopic dermatitis, allergic contact dermatitis, urticaria; vasculitis (e.g., necrotizing, cutaneous, and hypersensitivity vasculitis); eosinphilic myotis, eosiniphilic fasciitis,; and cancers.

In certain embodiments of the invention, the methods and compositions can be administered so as activate or promote chemokine activity or function for the treatment of diseases that are associated with immunosuppression such as individuals undergoing chemotherapy, radiation therapy, enhanced wound healing and bum treatment, therapy for autoimmune disease or other drug therapy (e.g., corticosteroid therapy) or combination of conventional drugs used in the treatment of autoimmune diseases and graft/transplantation rejection, which causes immunosuppression; immunosuppression due to congenital deficiency in receptor function or other causes; and infectious diseases, such as parasitic diseases, including but not limited to helminth infections, such as nematodes (round worms); Trichuriasis, Enterobiasis, Ascariasis, Hookworm, Strongyloidiasis, Trichinosis, filariasis; trematodes; visceral worms, visceral larva migtrans (e.g., Toxocara), eosinophilic gastroenteritis (e.g., Anisaki spp., Phocanema ssp.), cutaneous larva migrans (Ancylostona braziliense, Ancylostoma caninum); the malaria-causing protozoan Plasmodium vivax, Human cytomegalovirus, Herpesvirus saimiri, and Kaposi's sarcoma herpesvirus, also known as human herpesvirus 8, and poxyirus Moluscum contagiosum.

In certain embodiments, the present invention provides methods of modulating chemokine function, including methods of activation or methods of inhibition, comprising contacting a cell with an aryl nitrone of the invention. The cell can be any cell susceptible to modulation of chemokine activity and can be in vitro, such as a cultured cell, or in vivo. In certain embodiments, the aryl nitrone is a purified aryl nitrone or in the form of a pharmaceutical composition of the invention comprising a purified aryl nitrone. Purified aryl nitrones are substantially free from impurities as understood by those of skill in the art, for instance 90%, 95% or 99% free of impurities.

Compounds of the present invention may be used in combination with any other active agents or pharmaceutical compositions where such combined therapy is useful for the treatment or prevention of multiple sclerosis or a related condition.

### 5.3 Methods and Compositions for Treating or Preventing Multiple Sclerosis And Related Conditions

The present invention provides compositions comprising aryl nitrones of the invention and methods of their use for treating or preventing multiple sclerosis or related conditions. Related conditions include demyelinating disorders and also include, for instance, central pontine myelinolysis, Guillain-Barre syndrome and leukodystrophy. In preferred embodiments, the aryl nitrones are according to formula II.

In the methods of treatment, the subject can be any mammalian subject presenting one or more symptoms associated with multiple sclerosis or a related condition according to the judgment of one of skill in the art. In the methods of prevention, the subject can be any mammalian subject at risk for such a condition. In particularly preferred embodiments, the subject is a primate or a human.

Compounds of the present invention may be used in combination with any other active agents or pharmaceutical compositions where such combined therapy is useful for the treatment or prevention of multiple sclerosis or a related condition.

### 5.4 Pharmaceutical Compositions

### 5.4.1 Pharmaceutical Compositions Comprising the Aryl Nitrones According to Formula I

The present invention provides pharmaceutical compositions for the treatment or prevention of a condition associated with chemokine function such as a chemokine mediated condition such as multiple sclerosis or a related condition in a subject. The compositions comprise one or more of the aryl nitrones described in detail below. The pharmaceutical compositions are useful in the methods of treating or preventing a chemokine mediated condition such as multiple sclerosis, or a related condition, as described above.

In certain embodiments, the pharmaceutical compositions of the invention comprise an aryl nitrone. For example, the aryl nitrone can be a 3,4,5-trisubstituted aryl nitrone. In preferred embodiments of the invention, the pharmaceutical compositions of the invention comprise aryl nitrones of formula I, or a prodrug or pharmaceutically acceptable salt or solvate thereof:

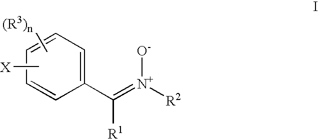

In formula I, any unsaturated carbon atom of the phenyl ring is replaced with a heteroatom to yield a heteroaryl ring. For instance, any unsaturated carbon atom of the phenyl ring of formula I can be replaced with a nitrogen atom. In some embodiments, none of the unsaturated carbon atoms of the phenyl ring of formula I is replaced with a heteroatom. In other embodiments, one, two or three of the unsaturated carbon atoms of the phenyl ring of formula I are replaced with a heteroatom to yield a heteroaryl ring. In preferred embodiments, the heteroatom is a nitrogen atom. In certain aspects, the present invention provides aryl nitrone compound according to formula I, wherein one, two or three of the unsaturated carbon atoms are replaced with a heteroatom as discussed herein.

X is —OH or any salt thereof.

R¹ is selected from the group consisting of hydrogen, alkyl, cycloalkyl and aryl. Preferably, R¹ is hydrogen or lower alkyl. More preferably, R¹ is hydrogen or alkyl having 1 to 4 carbon atoms, more preferably 1 or 2 carbon atoms. Still more preferably, R¹ is hydrogen.

R² is selected from the group consisting of alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heterocycloalkyl, substituted heterocycloalkyl, heterocycloalkenyl, substituted heterocycloalkenyl, heteroaryl, substituted heteroaryl, benzyl and substituted benzyl. R² is preferably selected from the group consisting of alkyl, substituted alkyl and cycloalkyl. More preferably, R² is alkyl having 3 to 6 carbon atoms or cycloalkyl having 5 to 6 carbon atoms. Particularly preferred R² groups include methyl, n-propyl, isopropyl, 1-hydroxy-2-methylprop-2-yl, n-butyl and tert-butyl.

The variable n is an integer from 1 to 4, and each R³ is independently selected from the group consisting of aryl, heteroaryl and the following formula: 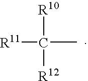
In certain embodiments, n is two. In particular embodiments, each R³ can be any aryl or heteroaryl group known to those of skill in the art including, groups with five or six members and bicyclic groups. Particular aryl and heteroaryl groups are described in the definitions above. In preferred embodiments, R³ has the above formula.

R¹⁰ is selected from the group consisting of hydrogen, lower alkyl and lower cycloalkyl; or R¹⁰ and R¹¹ can be joined to form an alkylene, substituted alkylene, or heteroalkylene group. R¹¹ and R¹² are independently selected from the group consisting of hydrogen, lower alkyl and lower cycloalkyl; or R¹¹ and R¹² can be joined to form an alkylene group having from 2 to 10 carbon atoms. Preferably, R¹⁰, R¹¹ and R¹² are independently lower alkyl. More preferably, R¹⁰, R¹¹ and R¹² are methyl.

In preferred embodiments of the invention, the pharmaceutical compositions of the invention comprise aryl nitrones of formula Ia, or a prodrug or pharmaceutically acceptable salt or solvate thereof:

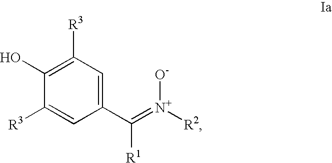

In formula Ia, any unsaturated carbon atom of the phenyl ring is replaced with a heteroatom to yield a heteroaryl ring. For instance, any unsaturated carbon atom of the phenyl ring of formula Ia can be replaced with a nitrogen atom. In some embodiments, none of the unsaturated carbon atoms of the phenyl ring of formula Ia is replaced with a heteroatom. In other embodiments, one, two or three of the unsaturated carbon atoms of the phenyl ring of formula Ia are replaced with a heteroatom to yield a heteroaryl ring. In preferred embodiments, the heteroatom is a nitrogen atom. In certain aspects, the present invention provides aryl nitrone compound according to formula Ia, wherein one, two or three of the unsaturated carbon atoms are replaced with a heteroatom as discussed herein.

R¹ is selected from the group consisting of hydrogen, alkyl, cycloalkyl and aryl. Preferably, R¹ is hydrogen or lower alkyl. More preferably, R¹ is hydrogen or alkyl having 1 to 4 carbon atoms, more preferably 1 or 2 carbon atoms. Still more preferably, R¹ is hydrogen.

R² is selected from the group consisting of alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heterocycloalkyl, substituted heterocycloalkyl, heterocycloalkenyl, substituted heterocycloalkenyl, heteroaryl, substituted heteroaryl, benzyl and substituted benzyl. R² is preferably selected from the group consisting of alkyl, substituted alkyl and cycloalkyl. More preferably, R² is alkyl having 3 to 6 carbon atoms or cycloalkyl having 5 to 6 carbon atoms. Particularly preferred R² groups include methyl, n-propyl, isopropyl, 1-hydroxy-2-methylprop-2-yl, n-butyl, tert-butyl and cyclohexyl.

Each R³is independently selected from the group consisting of aryl, heteroaryl and the following formula: 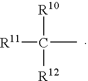
In certain embodiments, each R³ can be any aryl or heteroaryl group known to those of skill in the art including, groups with five or six members and bicyclic groups. Particular aryl and heteroaryl groups are described in the definitions above. In preferred embodiments, R³ has the above formula.

R¹⁰ is selected from the group consisting of hydrogen, lower alkyl and lower cycloalkyl; or R¹⁰ and R¹¹ can be joined to form an alkylene, substituted alkylene, or heteroalkylene group. R¹¹ and R¹² are independently selected from the group consisting of hydrogen, lower alkyl and lower cycloalkyl; or R¹¹ and R¹² can be joined to form an alkylene group having from 2 to 10 carbon atoms. Preferably, R¹⁰, R¹¹ and R¹² are independently lower alkyl. More preferably, R¹⁰, R¹¹ and R¹² are methyl.

In certain embodiments, at least one of R¹⁰, R¹¹ and R¹² is other than hydrogen. In further embodiments, at least two of R¹⁰, R¹¹ and R¹² is other than hydrogen. In particular embodiments, all three of R¹⁰, R¹¹ and R¹² is other than hydrogen.

In certain embodiments, one R³ is methyl and the other R³is tert-butyl. In other embodiments, each R³ group is methyl. In further embodiments, each R³ group is cyclohexyl. In further embodiments, each R³ group is propyl. For instance, in certain of the embodiments, each R³ group is isopropyl.

In one particular embodiment, R¹ is hydrogen, R² is tert-butyl and each R³ is tert-butyl, i.e. the aryl nitrone is α-(4-hydroxy-3,5-di-tert-butylphenyl)-N-tert-butylnitrone (or 2,6-di-tert-butyl-4-(N-tert-butyl) nitronyl phenol).

In a further embodiment, the pharmaceutical compositions useful in the invention contain or comprise a compound of formula Ib:

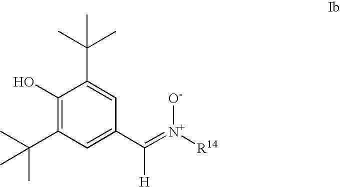

wherein R² is selected from the group consisting of alkyl, substituted alkyl, cycloalkyl, cycloheteroalkyl, substituted cycloalkyl, and arylalkyl;

and a prodrug, pharmaceutically-acceptable salt or solvate thereof.

R² is preferably selected from the group consisting of alkyl, substituted alkyl and cycloalkyl. More preferably, R² is alkyl having 3 to 6 carbon atoms or cycloalkyl having 5 to 6 carbon atoms. Particularly preferred R² groups include methyl, n-propyl, isopropyl, 1-hydroxy-2-methylprop-2-yl, n-butyl, tert-butyl, 3-thiomethylpropyl, 3-(thiomethoxy)but-1-yl, cyclohexyl, 4-trifluoromethybenzyl and 3,4,5-trimethoxybenzyl.

### 5.4.2 Pharmaceutical Compositions Comprising the Aryl Nitrones According to Formula II

The present invention provides pharmaceutical compositions for the treatment or prevention of multiple sclerosis or a related condition in a subject. The compositions comprise one or more of the aryl nitrones described in detail below. The pharmaceutical compositions are useful in the methods of treating or preventing a multiple sclerosis, or a related condition, as described above.

In certain embodiments, the pharmaceutical compositions of the invention comprise an aryl nitrone. For example, the aryl nitrone can be a 3,4,5-trisubstituted aryl nitrone. In preferred embodiments of the invention, the pharmaceutical compositions of the invention comprise aryl nitrones of formula II, or a prodrug, pharmaceutically-acceptable salt or solvate thereof:

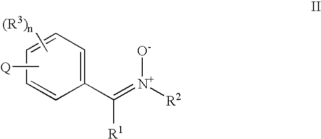

In certain embodiments according to formula II, any unsaturated carbon atom of the phenyl ring is replaced with a heteroatom to yield a heteroaryl ring. For instance, any unsaturated carbon atom of the phenyl ring of formula I can be replaced with a nitrogen atom. In some embodiments, none of the unsaturated carbon atoms of the phenyl ring of formula II is replaced with a heteroatom. In other embodiments, one, two or three of the unsaturated carbon atoms of the phenyl ring of formula II are replaced with a heteroatom to yield a heteroaryl ring. In preferred embodiments, the heteroatom is a nitrogen atom. In certain aspects, the present invention provides aryl nitrone compound according to formula II, wherein one, two or three of the unsaturated carbon atoms are replaced with a heteroatom as discussed herein.

Q is —OR, and R is selected from the group consisting of: 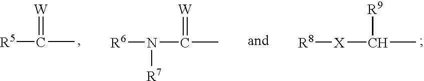
X is oxygen, sulfur, —S(O)— or —S(O)₂—, and W is oxygen or sulfur.

R¹ is selected from the group consisting of hydrogen, alkyl, cycloalkyl and aryl. Preferably, R¹ is hydrogen or lower alkyl. More preferably, R¹ is hydrogen or alkyl having 1 to 4 carbon atoms, more preferably 1 or 2 carbon atoms. Still more preferably, R¹ is hydrogen.

R² is selected from the group consisting of alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heterocycloalkyl, substituted heterocycloalkyl, heterocycloalkenyl, substituted heterocycloalkenyl, heteroaryl, substituted heteroaryl, benzyl and substituted benzyl. R² is preferably selected from the group consisting of alkyl, substituted alkyl and cycloalkyl. More preferably, R² is alkyl having 3 to 6 carbon atoms or cycloalkyl having 5 to 6 carbon atoms. Particularly preferred R² groups include methyl, n-propyl, isopropyl, 1-hydroxy-2-methylprop-2-yl, n-butyl, tert-butyl and cyclohexyl.

The variable n is an integer from 1 to 4, and each R³ is independently selected from the group consisting of aryl, heteroaryl and the following formula: 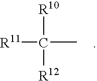
In certain embodiments, n is two. In particular embodiments, each R³ can be any aryl or heteroaryl group known to those of skill in the art including, groups with five or six members and bicyclic groups. Particular aryl and heteroaryl groups are described in the definitions above. In preferred embodiments, R³ has the above formula.

R¹⁰ is selected from the group consisting of hydrogen, lower alkyl and lower cycloalkyl; or R¹⁰ and R¹¹ can be joined to form an alkylene, substituted alkylene, or heteroalkylene group. R¹¹ and R¹² are independently selected from the group consisting of hydrogen, lower alkyl and lower cycloalkyl; or R¹¹ and R¹² can be joined to form an alkylene group having from 2 to 10 carbon atoms. Preferably, R¹⁰, R¹¹ and R¹² are independently lower alkyl. More preferably, R¹⁰, R¹¹ and R¹² are methyl.

R⁵ is selected from the group consisting of alkyl, substituted alkyl, alkenyl, substituted alkenyl, alkynyl, substituted alkynyl, cycloalkyl, substituted cycloalkyl, cycloalkenyl and substituted cycloalkenyl. R⁵ is preferably selected from the group consisting of alkyl and cycloalkyl. More preferably, R⁵ is lower alkyl. Particularly preferred R⁵ groups include methyl, ethyl, n-propyl, isopropyl and n-butyl.

R⁶ and R⁷ are independently selected from the group consisting of hydrogen, alkyl, substituted alkyl, alkenyl, substituted alkenyl, alkynyl, substituted alkynyl, cycloalkyl, substituted cycloalkyl, cycloalkenyl and substituted cycloalkenyl. In certain embodiments, R⁶ and R⁷ can be joined to form an alkylene or substituted alkylene group having from 2 to 10 carbon atoms. R⁶ is preferably selected from the group consisting of alkyl and alkoxycarbonylalkyl (i.e., ROC(O)-alkyl-, where R is alkyl or cycloalkyl). Particularly preferred R⁶ groups include ethyl, n-propyl, isopropyl, n-butyl, ethoxycarbonylmethyl and 2-(ethoxycarbonyl)ethyl. R⁷ is preferably hydrogen.

R⁸ is selected from the group consisting of alkyl, substituted alkyl, alkenyl, substituted alkenyl, alkynyl, substituted alkynyl, cycloalkyl, substituted cycloalkyl, cycloalkenyl and substituted cycloalkenyl. Preferably, R⁸ is alkyl or alkoxyalkyl (i.e., RO-alkyl-, where R is alkyl). Particularly preferred R⁸ groups include methyl and methoxyethyl. R⁹ is preferably hydrogen. Preferably, X is oxygen.

R⁹ is selected from the group consisting of hydrogen, alkyl, substituted alkyl, alkenyl, substituted alkenyl, alkynyl, substituted alkynyl, cycloalkyl, substituted cycloalkyl, cycloalkenyl and substituted cycloalkenyl. In certain embodiments, R⁹ can be joined to form an alkylene or substituted alkylene group having from 2 to 10 carbon atoms. In preferred embodiments, R⁹ is hydrogen.

In preferred embodiments of the invention, the pharmaceutical compositions of the invention comprise aryl nitrones of formula IIa, or a prodrug, pharmaceutically-acceptable salt or solvate thereof:

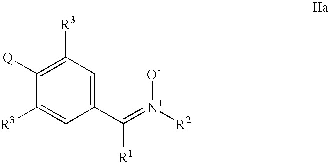

In certain embodiments according to formula IIa, any unsaturated carbon atom of the phenyl ring is replaced with a heteroatom to yield a heteroaryl ring. For instance, any unsaturated carbon atom of the phenyl ring of formula IIa can be replaced with a nitrogen atom. In some embodiments, none of the unsaturated carbon atoms of the phenyl ring of formula IIa is replaced with a heteroatom. In other embodiments, one, two or three of the unsaturated carbon atoms of the phenyl ring of formula IIa are replaced with a heteroatom to yield a heteroaryl ring. In preferred embodiments, the heteroatom is a nitrogen atom. In certain aspects, the present invention provides aryl nitrone compound according to formula IIa, wherein one, two or three of the unsaturated carbon atoms are replaced with a heteroatom as discussed herein.

Q is —OR, and R is selected from the group consisting of: 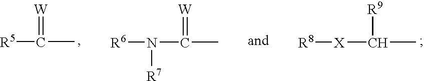
X is oxygen, sulfur, —S(O)— or —S(O)₂—, and W is oxygen or sulfur.

R¹ is selected from the group consisting of hydrogen, alkyl, cycloalkyl and aryl. Preferably, R¹ is hydrogen or lower alkyl. More preferably, R¹ is hydrogen or alkyl having 1 to 4 carbon atoms, more preferably 1 or 2 carbon atoms. Still more preferably, R¹ is hydrogen.

R² is selected from the group consisting of alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heterocycloalkyl, substituted heterocycloalkyl, heterocycloalkenyl, substituted heterocycloalkenyl, heteroaryl, substituted heteroaryl, benzyl and substituted benzyl. R² is preferably selected from the group consisting of alkyl, substituted alkyl and cycloalkyl. More preferably, R² is alkyl having 3 to 6 carbon atoms or cycloalkyl having 5 to 6 carbon atoms. Particularly preferred R² groups include methyl, n-propyl, isopropyl, 1-hydroxy-2-methylprop-2-yl, n-butyl, tert-butyl and cyclohexyl.

Each R³ is independently selected from the group consisting of aryl, heteroaryl and the following formula: 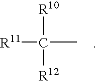
In certain embodiments, each R³ can be any aryl or heteroaryl group known to those of skill in the art including, groups with five or six members and bicyclic groups. Particular aryl and heteroaryl groups are described in the definitions above. In preferred embodiments, R³ has the above formula.

R¹⁰ is selected from the group consisting of hydrogen, lower alkyl and lower cycloalkyl; or R¹⁰ and R¹¹ can be joined to form an alkylene, substituted alkylene, or heteroalkylene group. R¹¹ and R¹² are independently selected from the group consisting of hydrogen, lower alkyl and lower cycloalkyl; or R¹¹ and R¹² can be joined to form an alkylene group having from 2 to 10 carbon atoms. Preferably, R¹⁰, R¹¹ and R¹² are independently lower alkyl. More preferably, R¹⁰, R¹¹ and R¹² are methyl.

R⁵ is selected from the group consisting of alkyl, substituted alkyl, alkenyl, substituted alkenyl, alkynyl, substituted alkynyl, cycloalkyl, substituted cycloalkyl, cycloalkenyl and substituted cycloalkenyl. R⁵ is preferably selected from the group consisting of alkyl and cycloalkyl. More preferably, R⁵ is lower alkyl. Particularly preferred R⁵ groups include methyl, ethyl, n-propyl, isopropyl and n-butyl.

R⁶ and R⁷ are independently selected from the group consisting of hydrogen, alkyl, substituted alkyl, alkenyl, substituted alkenyl, alkynyl, substituted alkynyl, cycloalkyl, substituted cycloalkyl, cycloalkenyl and substituted cycloalkenyl. In certain embodiments, R⁶ and R⁷ can be joined to form an alkylene or substituted alkylene group having from 2 to 10 carbon atoms. R⁶ is preferably selected from the group consisting of alkyl and alkoxycarbonylalkyl (i.e., ROC(O)-alkyl-, where R is alkyl or cycloalkyl). Particularly preferred R⁶ groups include ethyl, n-propyl, isopropyl, n-butyl, ethoxycarbonylmethyl and 2-(ethoxycarbonyl)ethyl. R⁷ is preferably hydrogen.

R⁸ is selected from the group consisting of alkyl, substituted alkyl, alkenyl, substituted alkenyl, alkynyl, substituted alkynyl, cycloalkyl, substituted cycloalkyl, cycloalkenyl and substituted cycloalkenyl. Preferably, R⁸ is alkyl or alkoxyalkyl (i.e., RO-alkyl-, where R is alkyl). Particularly preferred R⁸ groups include methyl and methoxyethyl. R⁹ is preferably hydrogen. Preferably, X is oxygen.

R⁹ is selected from the group consisting of hydrogen, alkyl, substituted alkyl, alkenyl, substituted alkenyl, alkynyl, substituted alkynyl, cycloalkyl, substituted cycloalkyl, cycloalkenyl and substituted cycloalkenyl. In certain embodiments, R⁹ can be joined to form an alkylene or substituted alkylene group having from 2 to 10 carbon atoms. In preferred embodiments, R⁹ is hydrogen.

In certain embodiments, at least one of R¹⁰, R¹¹ and R¹² is other than hydrogen. In further embodiments, at least two of R¹⁰, R¹¹ and R¹² is other than hydrogen. In particular embodiments, all three of R¹⁰, R¹¹ and R¹² are other than hydrogen.

In certain embodiments, one R³ is methyl and the other R³ is tert-butyl. In other embodiments, each R³ group is methyl. In further embodiments, each R³ group is cyclohexyl. In further embodiments, each R³ group is propyl. For instance, in certain of the embodiments, each R³ group is isopropyl.

In a further embodiment, the pharmaceutical compositions useful in the invention contain or comprise a compound of formula IIb: 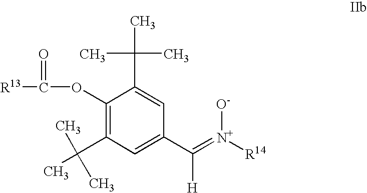
wherein
R¹³ is selected from the group consisting of alkyl, substituted alkyl, cycloalkyl and substituted cycloalkyl;
R¹⁴ is selected from the group consisting of alkyl, cycloalkyl, cycloheteroalkyl, substituted cycloalkyl, and arylalkyl;
and a prodrug, pharmaceutically-acceptable salt or solvate thereof.

Preferably, R¹³ is lower alkyl.

R¹⁴ is preferably selected from the group consisting of alkyl, substituted alkyl and cycloalkyl. More preferably, R¹⁴ is alkyl having 3 to 6 carbon atoms or cycloalkyl having 5 to 6 carbon atoms. Particularly preferred R¹⁴ groups include methyl, n-propyl, isopropyl, 1-hydroxy-2-methylprop-2-yl, n-butyl, tert-butyl, 3-thiomethylpropyl, 3-(thiomethoxy)but-1-yl, cyclohexyl, 4-trifluoromethybenzyl and 3,4,5-trimethoxybenzyl.

In another embodiment, the pharmaceutical compositions useful in the invention contain or comprise a compound of formula IIc: 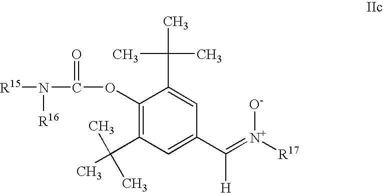
wherein
R¹⁵ and R¹⁶ are independently selected from the group consisting of hydrogen, alkyl, substituted alkyl, cycloalkyl, substituted cycloalkyl; or R¹⁵ and R¹⁶ can be joined to form an alkylene or substituted alkylene group having from 2 to 10 carbon atoms;
R¹⁷ is selected from the group consisting of alkyl, substituted alkyl, cycloalkyl, cycloheteroalkyl, substituted cycloalkyl, and arylalkyl;
and a prodrug, pharmaceutically-acceptable salt or solvate thereof.

R¹⁵ is preferably selected from the group consisting of alkyl and alkoxycarbonylalkyl (i.e., ROC(O)-alkyl-, where R is alkyl or cycloalkyl). Particularly preferred R¹⁵ groups include ethyl, n-propyl, isopropyl, n-butyl, ethoxycarbonylmethyl and 2-(ethoxycarbonyl)ethyl. R¹⁶ is preferably hydrogen.

R¹⁷ is preferably selected from the group consisting of alkyl, substituted alkyl and cycloalkyl. More preferably, R¹⁷ is alkyl having 3 to 6 carbon atoms or cycloalkyl having 5 to 6 carbon atoms. Particularly preferred R¹⁷ groups include methyl, n-propyl, isopropyl, 1-hydroxy-2-methylprop-2-yl, n-butyl, tert-butyl, 3-thiomethylpropyl, 3-(thiomethoxy)but-1-yl, cyclohexyl, 4-trifluoromethybenzyl and 3,4,5-trimethoxybenzyl.

In still another embodiment, the pharmaceutical compositions of the invention contain or comprise a compound of formula IId: 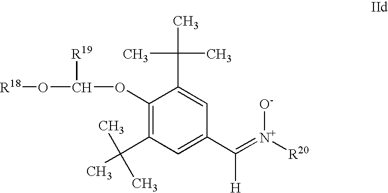
wherein
R¹⁸ is selected from the group consisting of alkyl, substituted alkyl, cycloalkyl and substituted cycloalkyl;
R¹⁹ is selected from the group consisting of hydrogen, alkyl, substituted alkyl, cycloalkyl and substituted cycloalkyl; or R¹⁸ and R¹⁹ can be joined to form an alkylene or substituted alkylene group having from 2 to 10 carbon atoms;
R²⁰ is selected from the group consisting of alkyl, substituted alkyl, cycloalkyl, cycloheteroalkyl, substituted cycloalkyl, and arylalkyl; and a prodrug, pharmaceutically-acceptable salt or solvate thereof.

Preferably, R¹⁸ is alkyl or alkoxyalkyl (i.e., RO-alkyl-, where R is alkyl). Particularly preferred R¹⁸ groups include methyl and methoxyethyl. R¹⁹ is preferably hydrogen.

R²⁰ is preferably selected from the group consisting of alkyl, substituted alkyl and cycloalkyl. More preferably, R²⁰ is alkyl having 3 to 6 carbon atoms or cycloalkyl having 5 to 6 carbon atoms. Particularly preferred R²⁰ groups include methyl, n-propyl, isopropyl, 1-hydroxy-2-methylprop-2-yl, n-butyl, tert-butyl, 3-thiomethylpropyl, 3-(thiomethoxy)but-1-yl, cyclohexyl, 4-trifluoromethybenzyl and 3,4,5-trimethoxybenzyl.

Particularly preferred 3,4,5-trisubstituted aryl nitrone compounds include those having the formulae shown in Tables I, II and III.

TABLE I

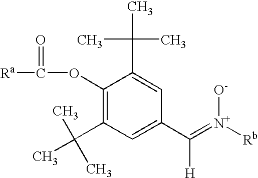

| Number | R^{a} | R^{b} |
|---|---|---|
| 2.1 | CH₃— | —C(CH₃)₃ |
| 2.2 | (CH₃)₂CH— | —C(CH₃)₃ |
| 2.3 | CH₃CH₂CH₂— | —C(CH₃)₃ |
| 2.4 | CH₃— | —CH(CH₃)₂ |
| 2.5 | CH₃— | —C(CH₃)₂CH₂OH |
| 2.6 | CH₃CH₂CH₂CH₂— | —C(CH₃)₃ |
| 2.7 | CH₃— | 4-CF₃-Ph-CH₂— |
| 2.8 | CH₃CH₂— | —C(CH₃)₃ |
| 2.9 | CH₃— | —CH₃ |
| 2.10 | CH₃— | 3,4,5-tri(CH₃O-)Ph-CH₂— |

TABLE II

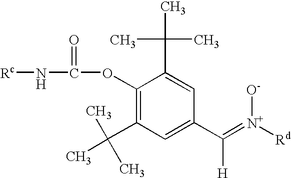

| Number | R^{c} | R^{d} |
|---|---|---|
| 2.11 | CH₃CH₂— | —C(CH₃)₃ |
| 2.12 | CH₃CH₂CH₂— | —C(CH₃)₃ |
| 2.13 | CH₃CH₂CH₂CH₂— | —C(CH₃)₃ |
| 2.14 | CH₃CH₂OC(O)CH₂CH₂— | —C(CH₃)₃ |
| 2.15 | CH₃CH₂OC(O)CH₂— | —C(CH₃)₃ |

TABLE III

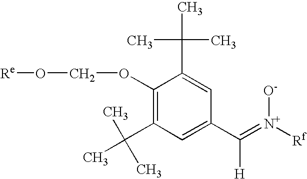

| Number | R^{e} | R^{f} |
|---|---|---|
| 2.16 | CH₃— | —C(CH₃)₃ |
| 2.17 | CH₃—O—CH₂CH₂— | —C(CH₃)₃ |
| 2.18 | CH₃— | —CH₂CH₂CH(SCH₃)CH₃ |
| 2.19 | CH₃— | —CH₂CH₂CH₂—SCH₃ |

Accordingly, in another of its composition aspects, the pharmaceutical compositions of the invention are prepared with each of the individual compounds:
α-(4-acetoxy-3,5-di-tert-butylphenyl)-N-tert-butylnitrone
α-(4-isobutanoyloxy-3,5-di-tert-butylphenyl)-N-tert-butylnitrone
α-(4-n-butanoyloxy-3,5-di-tert-butylphenyl)-N-tert-butylnitrone
α-(4-acetoxy-3,5-di-tert-butylphenyl)-N-isopropylnitrone
α-(4-acetoxy-3,5-di-tert-butylphenyl)-N-1-hydroxy-2-methylprop-2-ylnitrone
α-(4-n-pentanoyloxy-3,5-di-tert-butylphenyl)-N-tert-butylnitrone
α-(4-acetoxy-3,5-di-tert-butylphenyl)-N-4-trifluoromethylbenzylnitrone
α-(4-propionyloxy-3,5-di-tert-butylphenyl)-N-tert-butylnitrone
α-(4-acetoxy-3,5-di-tert-butylphenyl)-N-methylnitrone
α-(4-acetoxy-3,5-di-tert-butylphenyl)-N-3,4,5-trimethoxybenzylnitrone
α-[4-(ethylaminocarbonyloxy)-3,5-di-tert-butylphenyl]-N-tert-butylnitrone
α-[4-(n-propylaminocarbonyloxy)-3,5-di-tert-butylphenyl]-N-tert-butylnitrone
α-[4-(n-butylaminocarbonyloxy)-3,5-di-tert-butylphenyl]-N-tert-butylnitrone
α-[4-(2-ethoxycarbonyl)ethylaminocarbonyloxy)-3,5-di-tert-butylphenyl]-N-tert-butylnitrone
α-[4-(2-ethoxycarbonyl)methylaminocarbonyloxy)-3,5-di-tert-butylphenyl]-N-tert-butylnitrone
α-(4-methoxymethoxy-3,5-di-tert-butylphenyl)-N-tert-butylnitrone
α-[4-(2-methoxy)ethoxymethoxy-3,5-di-tert-butylphenyl]-N-tert-butylnitrone
α-(4-methoxymethoxy-3,5-di-tert-butylphenyl)-N-3-(thiomethoxy)but-1-ylnitrone
α-(4-methoxymethoxy-3,5-di-tert-butylphenyl)-N-3-thiomethoxypropylnitrone
and pharmaceutically acceptable salts thereof.

In one particular embodiment, R¹ is hydrogen, R² is tert-butyl, each R³ is tert-butyl, and Q is methoxymethoxy, i.e. the aryl nitrone is α-(4-methoxymethoxy-3,5-di-tert-butylphenyl)-N-tert-butylnitrone (or 3,5-di-tert-butyl-4-(methoxymethoxy)phenol-(N-tert-butyl) nitrone).

### 5.4.3 Pharmaceutical Compositions Comprising the Aryl Nitrones of the Invention

Generally, the aryl nitrones of this invention are administered in a pharmaceutically effective amount. In preferred embodiments, the aryl nitrone is according to formula I or formula II. The amount of the aryl nitrone administered will typically be determined by a physician, in the light of the relevant circumstances, including the condition to be treated and the severity thereof, the chosen route of administration, the compound administered, the age, weight, and co-morbid conditions of the individual subject.

When employed as pharmaceuticals, the aryl nitrones of this invention are typically administered in the form of a pharmaceutical composition. Such compositions can be prepared using procedures well known in the pharmaceutical art and comprise at least one active compound. As is known to those of skill in the art, in most pharmaceutical compositions, the active agent, (the aryl nitrones) is usually a minor component (from about 0.1 to about 50% by weight or preferably from about 1 to about 40% by weight) with the remainder being various vehicles, carriers and other excipients or processing aids helpful for forming the desired dosing form.

The pharmaceutical compositions typically include a pharmaceutically acceptable carrier. Pharmaceutically acceptable carriers include, for example, normal saline, glucose, trehalose, sucrose, sterile water, buffered water, 0.4% saline, and 0.3% glycine. Pharmaceutical compositions often include a pharmaceutically acceptable buffer. Suitable buffers include, but are not limited to, citrate, acetate, phosphate, tris(hydroxymethyl)-aminomethane or THAM (tromethamine). The compositions can contain pharmaceutically acceptable excipients or auxiliary substances as required to approximate physiological conditions, such as pH adjusting and buffering agents, and tonicity adjusting agents, for example, sodium acetate, sodium lactate, sodium chloride, potassium chloride, calcium chloride and the like. Injectable pharmaceutical compositions often include an antimicrobial, especially for multi-dose dosage forms.

Injectable compositions, can be sterilized by conventional, well known sterilization techniques. The resulting aqueous solutions may be packaged for use or filtered under aseptic conditions and lyophilized, the lyophilized preparation being combined with a sterile aqueous solution prior to administration.

The pharmaceutical compositions of this invention can be administered by any suitable route including, by way of illustration, oral, topical, rectal, transdermal, subcutaneous, intravenous, intramuscular, intranasal, and the like. Depending on the intended route of delivery, the compounds of this invention are preferably formulated as either oral, topical or injectable compositions.

Pharmaceutical compositions for oral administration can take the form of bulk liquid solutions or suspensions, or bulk powders. More commonly, however, such compositions are presented in tablet or capsule form to facilitate, for example, accurate dosing.

For oral administration, the pharmaceutical formulations can take the form of, for example, tablets or capsules prepared by conventional means with pharmaceutically acceptable excipients such as binding agents (e.g., pregelatinised maize starch, polyvinylpyrrolidone, hydroxypropyl methylcellulose, microcrystalline cellulose, gum tragacanth or gelatin); fillers (e.g., lactose, microcrystalline cellulose or calcium hydrogen phosphate); lubricants ( e.g., magnesium stearate, talc or silica); disintegrants (e.g., potato starch, corn starch, sodium starch, glycolate, alginic acid or primogel); wetting agents (e.g., sodium lauryl sulfate); sweetening agents (e.g. sucrose or saccharin) or flavoring agents (e.g. peppermint or orange flavoring). The tablets can be coated by methods well known in the art (see, Remington's Pharmaceutical Sciences, 18th Edition Gennaro et al., eds.) Mack Printing Company, Easton, Pa., 1990, incorporated herein by reference in its entirety).

Liquid aryl nitrone pharmaceutical compositions for oral administration can take the form of, for example, solutions, syrups or suspensions, or they can be a dry product for constitution with water or other suitable vehicle before use. Such liquid pharmaceutical compositions can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid).

The term oral refers to a route of administration via the mouth as well as buccal, rectal and vaginal routes of administration. Buccal administration, for example, in the form of tablets, troches or lozenges is contemplated. For rectal and vaginal routes of administration, the aryl nitrones can be prepared as solutions (e.g., for retention enemas) suppositories or ointments. Topical administration refers to any route of administration through the skin, including, but not limited to, creams, ointments, gels and transdermal patches (see, Remington's Pharmaceutical Sciences, supra). Injectable administration refers to any route of administration that is not topical or through the alimentary canal. Injectable pharmaceutical compositions of the present invention can be administered, for example, into a vein (intravenously), an artery (intraarterially), a muscle (intramuscularly), under the skin (subcutaneously) or into a joint (intraarticular).

Injectable pharmaceutical compositions can be sterile suspensions, solutions or emulsions of aryl nitrones in aqueous or oily vehicles. The compositions can also comprise formulating agents or excipients, such as suspending, stabilizing and/or dispersing agent. Depot or sustained release pharmaceutical compositions can be used in the methods of the invention. For example, the aryl nitrones can be formulated with a suitable polymeric or hydrophobic materials (e.g., as an emulsion in an acceptable oil) to provide extended or sustained release of the aryl nitrones.

In certain embodiments, the aryl nitrone pharmaceutical compositions can be transdermal delivery systems manufactured as an adhesive disc or patch, (e.g. reservoir, porous membrane type or solid matrix) that slowly release the aryl nitrone for percutaneous absorption. To this end, permeation enhancers can be used to facilitate transdermal penetration of the aryl nitrones.

In certain embodiments, the aryl nitrone pharmaceutical compositions can be topical creams, lotions, gels, ointments and the like. Topical compositions are typically formulated as a topical ointment or cream containing the active ingredient(s), generally in an amount ranging from about 0.01 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example, an oil-in-water cream base. Such topical formulations are well-known in the art and generally include additional ingredients to enhance the dermal penetration or stability of the active ingredients or the formulation. All such known topical formulations and ingredients are included within the scope of this invention.

In another embodiment, the pharmaceutical compositions can be in unit dose or unit of use forms or packages. As is known to those of skill in the art, a unit dose form or package is a convenient, prescription size, patient ready unit labeled for direct distribution by health care providers. A unit of use form contains a pharmaceutical composition in an amount necessary for a typical treatment interval and duration for a given indication.

A unit dosage form contains a pharmaceutical composition in an amount necessary for administration of a single dose of the composition. The present invention provides unit dosage forms of pharmaceutical compositions in an amount for delivery of a dose of about 1 to 25 mg/kg, about 1 to 20 mg/kg, about 1 to 15 mg/kg, about 1 to 10 mg/kg, about 1 to 7.5 mg/kg, about 1 to 5 mg/kg of the aryl nitrone to a subject. The subject can be, for example, a human subject with an average weight of about 70 kg. In certain embodiments, the present invention provides a unit dosage form that comprises about 1 to 1750 mg, about 1 to 1500 mg, about 1 to 1250 mg, about 1 to 1000 mg, about 1 to 750 mg, about 250 to 750 mg, or about 500 to 750 mg of the aryl nitrone. In certain embodiments, the unit dosage form consists essentially of these amounts of the aryl nitrone; in other words, the unit dosage form can additionally comprise other ingredients for administration of the aryl nitrone such as pharmaceutically acceptable carrier, excipient or diluent, a vial, syringe, or patch or other ingredients known to those of skill in the art for administering the aryl nitrone.

Typical unit dosage forms include prefilled, premeasured ampules or syringes of the injectable compositions or unit dose wrapped tablets or capsules in the case of solid, oral compositions The unit dosage form can be, for example, a single use vial, a pre-filled syringe, a single transdermal patch and the like.

As is known to those of skill in the art, a unit of use form or package is a convenient, prescription size, patient ready unit labeled for direct distribution by health care providers. A unit of use form contains a pharmaceutical composition in an amount necessary for a typical treatment interval and duration for a given indication. The methods of the invention provide for a unit-of-use package of a pharmaceutical composition comprising, for example, an aryl nitrone in an amount sufficient to treat an average sized adult male or female with about 250 mg subcutaneously three times weekly for one month. Thus a unit of use package as described above would have twelve (three times per week injections for four weeks) prefilled syringes each containing 250 mg of aryl nitrone pharmaceutical composition.

The pharmaceutical compositions in a container can be labeled and can have accompanying labeling or written information to identify the composition contained therein and other information useful to health care providers and subjects in the treatment of multiple sclerosis, including, but not limited to, instructions for use, dose, dosing interval, duration, indication, contraindications, warnings, precautions, handling and storage instructions and the like. In certain embodiments, the present invention provides kits comprising a pharmaceutical composition of the invention in a container and written information, such as a label or labeling, to identify the composition contained therein and other information useful to health care providers and subjects in the treatment of multiple sclerosis, including, but not limited to, instructions for use, dose, dosing interval, duration, indication, contraindications, warnings, precautions, handling and storage instructions and the like.

The following formulation examples illustrate representative pharmaceutical compositions of this invention. The present invention, however, is not limited to the following pharmaceutical compositions.

### 5.4.3.1 Formulation 1—Tablets

A compound of formula I or II is admixed as a dry powder with a dry gelatin binder in an approximate 1:2 weight ratio. A minor amount of magnesium stearate is added as a lubricant. The mixture is formed into 240-270 mg tablets (80-90 mg of active nitrone compound per tablet) in a tablet press.

### 5.4.3.2 Formulation 2—Capsules

A compound of formula I or II is admixed as a dry powder with a starch diluent in an approximate 1:1 weight ratio. The mixture is filled into 250 mg capsules (125 mg of active nitrone compound per capsule).

5.4.3.3 Formulation 3—Liquid

A compound of formula I or II (125 mg), sucrose (1.75 g) and xanthan gum (4 mg) are blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 mL.

### 5.4.3.4 Formulation 4—Injection

The compound of formula I or II is dissolved in a buffered sterile saline injectable aqueous medium to a concentration of approximately 5 mg/mL.

### 5.4.3.5 Formulation 5—Ointment

Stearyl alcohol (250 g) and white petrolatum (250 g) are melted at about 75° C. and then a mixture of a compound of formula I (50 g), methylparaben (0.25 g), propylparaben (0.15 g), sodium lauryl sulfate (10 g), and propylene glycol (120 g) dissolved in water (about 370 g) is added and the resulting mixture is stirred until it congeals.

### 5.5 Methods of Making the Aryl Nitrones

The aryl nitrones of this invention can be prepared from readily available starting materials using the following general methods and procedures, for instance, those described in detail in U.S. Pat. No. 6,342,523, the contents of which are hereby incorporated by reference in their entirety. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in THEODORA. W. GREENE & PETER G. M. WUTS, PROTECTING GROUPS IN ORGANIC SYNTHESIS (2d ed. 1991), and references cited therein.

### 5.5.1 Methods of Making the Aryl Nitrones of Formula I

In one preferred method of synthesis, the aryl nitrones of Formula I are prepared by coupling an aryl carbonyl compound with a hydroxylamine: 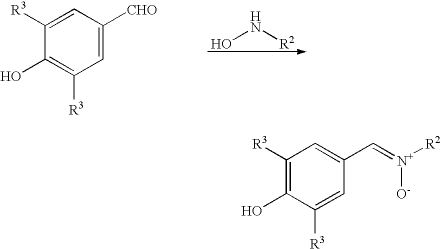
For example, compound 1, that is, α-(4-hydroxy-3,5-di-tert-butylphenyl)-N-tert-butylnitrone can be prepared as follows: 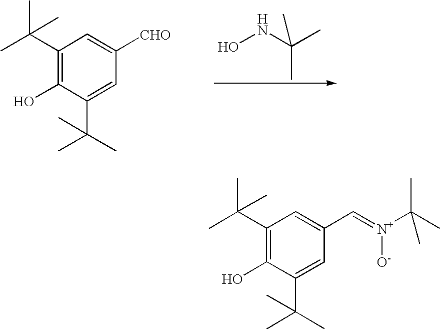
This coupling reaction can typically be conducted by contacting the aryl carbonyl compound with at least one equivalent, preferably about 1.1 to about 2 equivalents, of the hydroxylamine in an inert polar solvent such as methanol, ethanol, 1,4-dioxane, tetrahydrofuran, dimethyl sulfoxide, dimethylformamide and the like. This reaction is preferably conducted at a temperature of from about 0° C. to about 100° C. for about 1 to about 48 hours or more. Optionally, a catalytic amount of an acid, such as hydrochloric acid, acetic acid, p-toluenesulfonic acid, silica gel and the like, may be employed in this reaction. Alternatively, the reaction times can be reduced substantially by subjecting the starting materials to microwave irradiation. Upon completion of the reaction, the aryl nitrone is recovered by conventional methods including precipitation, chromatographic separation, filtration, distillation, sublimation, and the like.

The aryl carbonyl compounds and hydroxyl amines employed in the above-described coupling reaction are either known or can be prepared from known compounds by conventional procedures.

Nitrones of formula I may also be prepared by alternative well-documented methods such as oxidation of amines, imines, hydroxylamines and N-alkylation of oximes. Exemplary schemes for compound 1 are illustrated below:

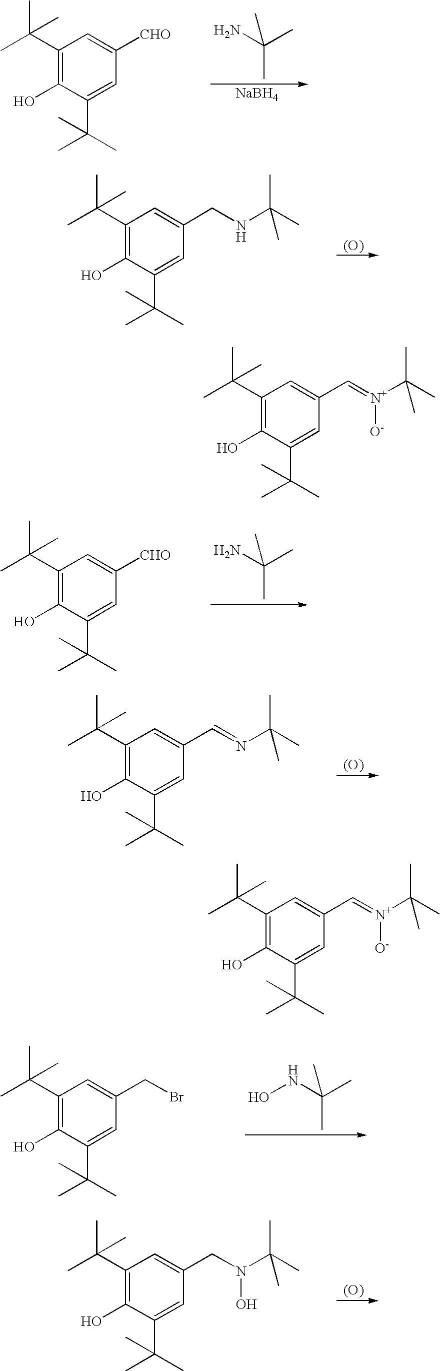

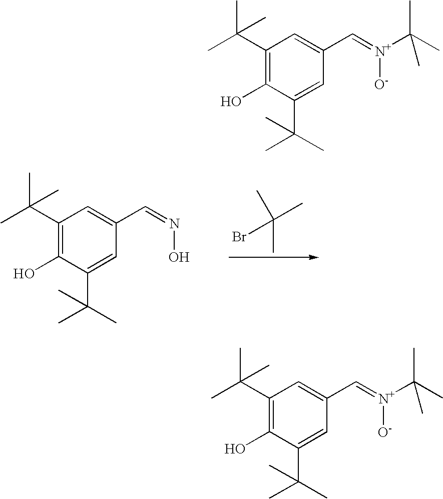

Other compounds of the invention are within the skill of those in the art given the above exemplary teaching, for example, by adjusting starting materials in the above schemes. In particular, one of skill in the art can readily prepare a compound of the invention having a hydroxyl in the “2” or ortho position by staring with the appropriate aryl carbonyl compound, etc.

### 5.5.2 Methods of Making the Aryl Nitrones of Formula II

In one preferred method of synthesis, the aryl nitrones of Formula II are prepared by coupling an aryl carbonyl compound with a hydroxylamine: 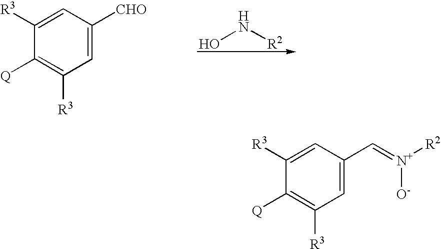
This coupling reaction can typically be conducted by contacting the aryl carbonyl compound with at least one equivalent, preferably about 1.1 to about 2 equivalents, of the hydroxylamine in an inert polar solvent such as methanol, ethanol, 1,4-dioxane, tetrahydrofuran, dimethyl sulfoxide, dimethylformamide and the like. This reaction is preferably conducted at a temperature of from about 0° C. to about 100° C. for about 1 to about 48 hours or more. Optionally, a catalytic amount of an acid, such as hydrochloric acid, acetic acid, p-toluenesulfonic acid, silica gel and the like, may be employed in this reaction. Alternatively, the reaction times can be reduced substantially by subjecting the starting materials to microwave irradiation. Upon completion of the reaction, the aryl nitrone is recovered by conventional methods including precipitation, chromatographic separation, filtration, distillation, sublimation, and the like.

The aryl carbonyl compounds and hydroxyl amines employed in the above-described coupling reaction are either known or can be prepared from known compounds by conventional procedures.

Other compounds of the invention are within the skill of those in the art given the above exemplary teaching, for example, by adjusting starting materials in the above schemes.

### 5.6 Methods of Administration

The compositions of the invention can be administered according to any method of administering a pharmaceutical composition known to those of skill in the art.

Unless described otherwise, an aryl nitrone of the invention, or compositions thereof, will generally be used in an amount effective to achieve the intended purpose. The compounds of the invention or compositions thereof, are administered or applied in a therapeutically effective amount for use to treat or prevent multiple sclerosis or related conditions as discussed above.

The amount of the aryl nitrone of the invention that will be effective in the treatment of a particular disorder or condition disclosed herein will depend on the nature of the disorder or condition, and can be determined by standard clinical techniques known in the art as previously described. In addition, in vitro or in vivo assays may optionally be employed to help identify optimal dosage ranges. The amount of a compound of the invention administered will, of course, be dependent on, among other factors, the subject being treated, the weight of the subject, the severity of the affliction, the manner of administration and the judgment of the prescribing physician.

For example, the dosage may be delivered in a pharmaceutical composition by a single administration, by multiple applications or controlled release. In a preferred embodiment, the compounds of the invention are delivered by oral sustained release administration. Preferably, in this embodiment, the compounds of the invention are administered twice per day (more preferably, once per day). Dosing may be repeated intermittently, may be provided alone or in combination with other drugs and may continue as long as required for effective treatment of the disease state or disorder.

Suitable dosage ranges for oral administration are dependent on the potency of the compound of the invention, but are generally about 0.001 mg to about 25 mg of a compound of the invention per kilogram body weight of the subject. Other useful dosage ranges include, for example, about 1 to 25 mg/kg, about 1 to 20 mg/kg, about 1 to 15 mg/kg, about 1 to 10 mg/kg, about 1 to 7.5 mg/kg, about 1 to 5 mg/kg of the aryl nitrone. The subject can be, for example, a human subject with an average weight of about 70 kg. Further dosage ranges may be readily determined by methods known to the skilled artisan. For certain embodiments of the invention, particular unit dosage forms are discussed in detail above.

As discussed above, the compounds described herein are suitable for use in a variety of drug delivery systems. Injection dose levels for treating conditions such as multiple sclerosis- related conditions range from about 0.1 mg/kg/hour to at least 10 mg/kg/hour from about 1 to about 120 hours to achieve a total dose of about 0.1 to about 25 mg/kg, as described above. A preloading bolus of from about 0.1 mg/kg to about 10 mg/kg or more may also be administered to achieve adequate steady state levels. Each maximum total dose is not expected to exceed about 1750 mg for a 40 to 80 kg human patient.

For the treatment of long-term conditions, the regimen for treatment may stretch over many months or years so oral dosing is preferred for patient convenience and tolerance. With oral dosing, one to five and especially two to four and typically three oral doses per day are representative regimens. Using these dosing patterns, each dose provides from about 0.1 to about 25 mg/kg or about 0.1 to about 20 mg/kg of the nitrone, with preferred doses each providing from about 0.1 to about 10 mg/kg and especially about 1 to about 5 mg/kg.

The compounds of the invention are preferably assayed in vitro and in vivo, for the desired therapeutic or prophylactic activity, prior to use in subjects. For example, in vitro assays can be used to determine whether administration of a specific compound of the invention or a combination of compounds of the invention is preferred for reducing one or more symptoms of multiple sclerosis. The compounds of the invention may also be demonstrated to be effective and safe using animal model systems.

Preferably, a therapeutically effective dose of a compound of the invention described herein will provide therapeutic benefit without causing substantial toxicity. Toxicity of compounds of the invention may be determined using standard pharmaceutical procedures and may be readily ascertained by the skilled artisan. The dose ratio between toxic and therapeutic effect is the therapeutic index. A compound of the invention will preferably exhibit particularly high therapeutic indices in treating disease and disorders. The dosage of a compound of the inventions described herein will preferably be within a range of circulating concentrations that include an effective dose with little or no toxicity.

The compounds of this invention can be administered as the sole active agent or they can be administered in combination with other appropriate active agents known to those of skill in the art to be useful for the treatment or prevention of multiple sclerosis or related conditions. For example, the compounds of the invention can be administered in combination with interferons such as AVONEX® (Biogen Idec, Cambridge, Mass.), BETASERON® (Berlex, Richmond, Calif.), NOVANTRONE®, REBIF® and REBIJECT® (Serono Inc., Geneva, Switzerland) or with COPAXONE® (Teva Pharmaceutical Industries Ltd., North Wales, Pa.).

The following synthetic and biological examples are offered to illustrate this invention and are not to be construed in any way as limiting the scope of this invention.

### 6. EXAMPLES

### 6.1 Example 1

### Synthesis of α-(4-hydroxy-3,5-di-tert-butylphenyl)-N-tert-butylnitrone (Compound 1)

To a well stirred solution of 3,5-di-tert-butyl-4-hydroxy-benzaldehyde (100 g, 0.41 mol) and N-tert-butyl hydroxylamine (65.0 g, 0.73 mol) in methanol (2.0 L) was added 10 drops of conc. HCl and the mixture was refluxed for 5 days. The mixture was concentrated to dryness and the residue was dissolved in 700 mL of ethyl acetate and left in a refrigerator overnight where upon the product crystallized out. The crystalline product was filtered and vacuum dried to obtain 116.95 g (93.4%) of the title compound.

### 6.2 Example 2

### Synthesis of α-(4-methoxymethoxy-3,5-di-tert-butyl-phenyl)-N-tert-butylnitrone (Compound 15)

To a solution of 3,5-di-tert-butyl-4-methoxymethoxybenzaldehyde (11.42 g, 40 mmol) in benzene (200 mL) was added tert-butylhydroxylamine (4.0 g, 50 mmol). The resulting solution was refluxed for 72 h until no more aldehyde was detected by TLC (R_{f}=0.56 for product and 0.78 for starting material in 1:1 hexanes/EtOAc). The solvent was removed in vacuo and the residue was suspended in hexane/EtOAc. The suspension was filtered, washed with hexanes and dried to afford the title compound (69% yield) as a white solid, m.p. 202.2-205.9° C. Spectroscopic data were as follows: ¹H NMR (DMSO-d₆, 270 MHz): δ=8.37 (2H, s, phenyl H), 7.79 (1H, s, nitronyl H), 4.84 (2H, s, OCH₂), 3.54 (3H, s, OCH₃), 1.48 (9H, s, 3 CH₃), 1.40 (18H, s, 6 CH₃). ¹³C NMR (DMSO-d₆, 270 MHz): δ=155.3, 143.9, 129.4, 127.8, 127.4, 101.0, 70.5, 57.6, 36.0, 32.3, 28.5.

### 6.3 Examples 3-18

### Exemplary Compounds of the Invention

Compounds 1-15 and 17, described in detail below, are prepared according to the methods of the invention.

| Entry | Structure | Mol. Formula | Mol. Weight |
|---|---|---|---|
| 1 | 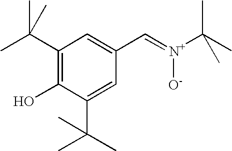 | C19H31NO2 | 305.46 |
| 2 | 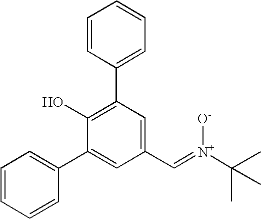 | C23H23NO2 | 345.43 |
| 3 | 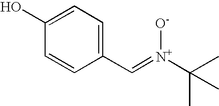 | C11H15NO2 | 193.24 |
| 4 | 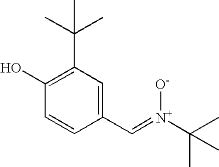 | C15H23NO2 | 249.35 |
| 5 | 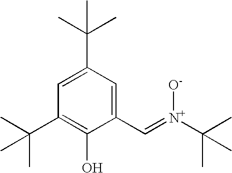 | C19H31NO2 | 305.45 |
| 6 | 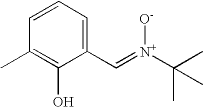 | C12H17NO2 | 207.27 |
| 7 | 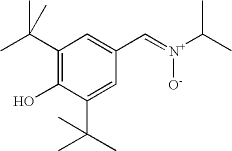 | C18H29NO2 | 291.44 |
| 8 | 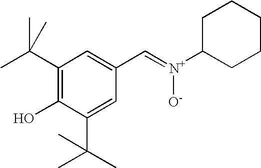 | C21H33NO2 | 331.50 |
| 9 | 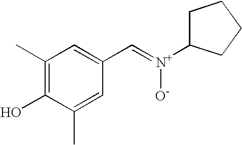 | C14H19NO2 | 233.31 |
| 10 | 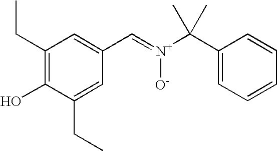 | C20H25NO2 | 311.43 |
| 11 | 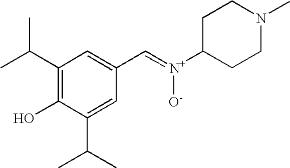 | C19H30N2O2 | 318.46 |
| 12 | 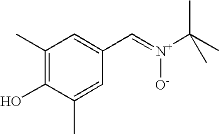 | C13H19NO2 | 221.30 |
| 13 | 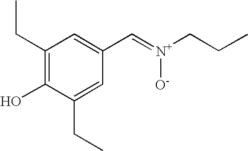 | C14H21NO2 | 235.33 |
| 14 | 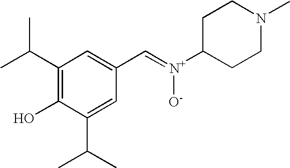 | C19H30N2O2 | 318.46 |
| 15 | 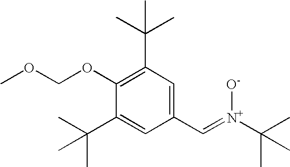 | C21H34NO3 | 348.51 |
| 17 | 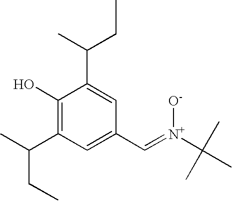 | C19H31NO2 | 305.45 |

### 6.4 Example 19

### Pretreatment with α-(4-hydroxy-3,5-di-tert-butylphenyl)-N-tert-butylnitrone (Compound 1) Inhibits HL-60 Cell Chemotaxis

The instant example demonstrates the effectiveness of the compositions and methods of the invention in modulating chemokine function. In particular, the instant example demonstrates that the methods and compositions of the invention are useful for the modulation of chemokine mediated cell migration in vitro. As is known to those of skill in the art, chemokine mediated cell migration is a key step in the progression of chemokine mediated diseases such as multiple sclerosis. See, e.g., Miller et al., 2003, N. Engl. J Med. 348:15-23; Glabinksi and Ransohoff, 1999, J. Neuro Virol. 5:623-634; Ransohoffet al., 2003, Nat. Rev. Immunol. 3:569-581.

Compound 1 was prepared according to the methods of the invention and tested as described below. Briefly, cells were pre-incubated with Compound 1, and then assayed for chemokine-mediated cell migration.

In vitro cell migration assays were performed using differentiated HL-60 cells. HL-60 cells were differentiated by adding 1.4% DMSO to RPMI-1640 media supplemented with 10% FBS and 100 μg/ml penicillin/streptomycin. Cells on the sixth day following differentiation were used for in vitro migration assays.

In vitro migration assays were performed using a Neuroprobe migration apparatus (MBB96, Neuro Probe, Inc., Gaithersburg, Md. 20877) according to the manufacturer's protocol with slight modifications. Briefly, about 370 μl of diluted chemokine or control was added to a 96-well fibronectin-coated, black-walled Packard plate. Chemoattractants included 100 nM formyl-methionyl-leucyl-phenylalanine (fMLP), 30 ng/ml chemokine Stromal Cell-Derived Factor-1 (SDF-1), 300 ng/ml chemokine IL-8. Negative control included migration media alone. The plate was placed into the migration apparatus, an 8 μm framed filter was carefully placed on top of the plate, and the apparatus was screwed down. The chemokines and control were allowed to equilibrate at 37° C. for 15 minutes.

Differentiated HL-60 cells were resuspended in complete media (RPMI-1640 media supplemented with 10% FBS and 100 μg/ml penicillin/streptomycin) at 4×10⁵ cells/ml. Cells were incubated in the presence of media alone or various concentrations of compound 1 for 30 minutes at 37° C. The cells were collected by centrifugation and were resuspended at 4×10⁵ cells/ml in migration media (RPMI-1640+0.1% BSA). 100 μl of cells was added to each of the top wells in the migration apparatus. Cells were allowed to migrate for 2 hours at 37° C.

After allowing migration, the apparatus was disassembled, and the Packard plate was centrifuged at 1500 rpm for 10 minutes in a tabletop centrifuge. Cells were allowed to adhere for 1 hour at 37° C.

Cells were then fixed for 20-30 minutes at ambient temperature by removing 180 μl of migration media and adding 180 μl of 8% paraformaldehyde.

After removing fixative solution, cells were washed with 200 μl phosphate-buffered saline for 5 minutes (using a multichannel Rainin LTS pipetman to minimize disruption). Cells were stained by the addition of Hoechst stain diluted 1:1000 in phosphate-buffered saline and incubation at ambient temperature for 15 minutes.

After staining, the cells were washed with 200 μl phosphate-buffered saline. Cell number in each well was quantitated using ImageXpress (Axon Instruments) or a fluorescent plate reader.

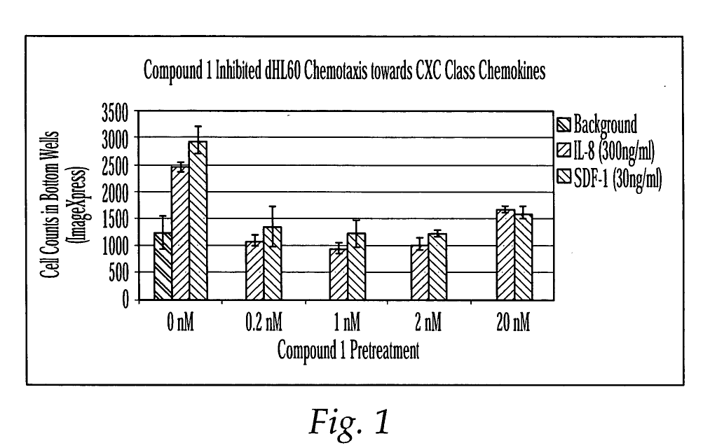

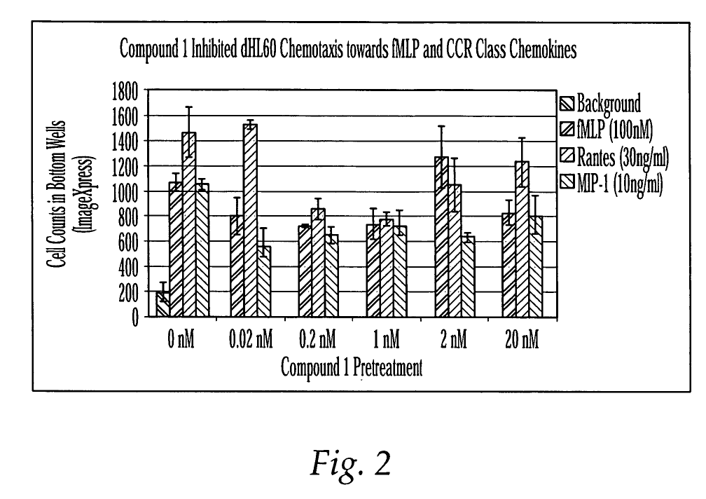

As shown in FIGS. 1 and 2, pretreatment of differentiated HL-60 cells with compound 1 inhibits chemokine mediated chemotaxis of the cells toward chemoattractants including fMLP, CXC chemokines such as IL-8 and SDF-1 and CCR chemokines such as Rantes and MIP-1. These results indicate that compound 1 is able to interfere with chemokine receptor mediated migration and thereby inhibit chemotaxis toward chemokines in this assay.

### 6.5 Example 20

### Compound 1 Induces Cell Migration of CHO-K1 Cells Expressing Various Chemokine Receptors

The instant example further demonstrates the effectiveness of the compositions and methods of the invention in modulating chemokine function. In particular, the instant example demonstrates that the methods and compositions of the invention are useful for the modulation of chemokine activity in cells expressing particular chemokine receptors.

CHO-K1 cells were obtained from American Type Culture Collection (CCL-61) and cultured in Ham's F12 media supplemented with 10% heat inactivated FBS and 100 μg/ml penicillin/streptomycin. cDNA fragments encompassing chemokine receptor coding regions were obtained from cDNAs synthesized from HL-60 cells and/or human peripheral blood mononuclear cells by the polymerase chain reaction. Expression constructs of chemokine receptors were generated by cloning PCR fragments into expression vector pEF6/V5-TOPO (K9610-20, Invitrogen). Transient transfections were carried out with Fugene 6 transfection reagent (1814443, Roche Applied Science) according to the manufacturer's instructions.

The CHO-chemokine receptor cells were used in migration assays 36 hours following transfection. In vitro migration assays were performed using a Neuroprobe Blindwell migration apparatus (AA12, Neuro Probe, Inc./16008 Industrial Drive, Gaithersburg, Md. 20877) according to the manufacturer's instructions with slight modifications. Briefly, cells were trypsinized from culture flasks and allowed to recover in complete medium for 2 hours. Cells were then resuspended to 4×10⁵ cells/ml in migration medium (Ham's F12+0.1% BSA). A chemokines, compound 1, or control was added to the bottom wells of the migration chamber. 100 μl of cells were added to each of the top wells in the migration apparatus. Cells were allowed to migrate for 2 hours at 37° C.

Following the migration assays, migrated cells that had adhered to the underside of the filter were quantitated according to the manufacturer's instructions. Briefly, the filters were carefully immersed in methanol and placed cell-side up on a disposable lint-free towel for air-drying. Air-dried filtered were stained in Diff-Quik (available from most major laboratory suppliers) according to manufacturer's instructions. Stained filtered were mounted onto microscope slides and migrated cells in each well were manually counted under 40× magnification.

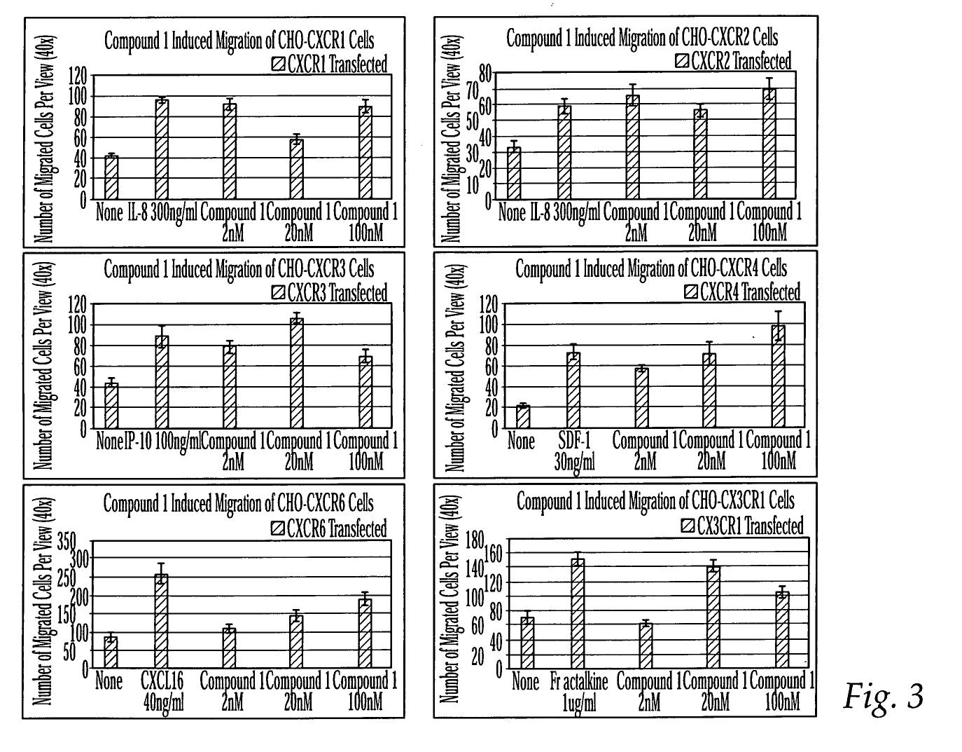

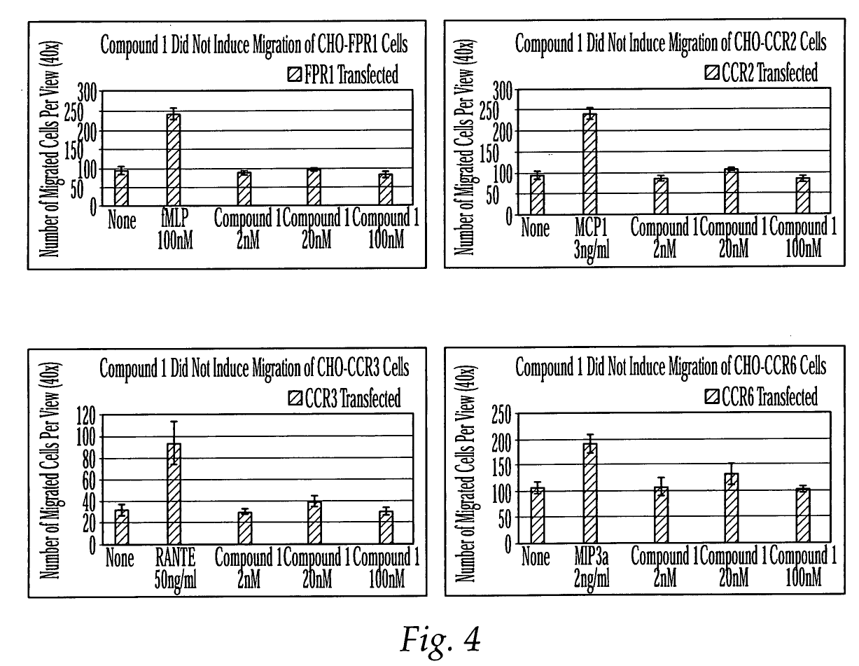

As shown in FIG. 3, compound 1 induced migration in CHO-K1 cells that expressed CXCR chemokine receptors. For instance, compound 1 induced migration of CHO-K1 cells expressing CXCR1 to a degree similar to the migration of the same cells induced by IL-8 (see FIG. 3, first panel). Similarly, compound 1 induced migration of CHO-K1 cells expressing CXCR2, CXCR3, CXCR4, CXCR6 or CX3CR1 (see FIG. 3, panels 2-6). Compound 1 did not induce migration of CHO-K1 cells expressing the FMLP receptor or CCR receptors (see FIG. 4).

### 6.6 Example 21

### Compounds 1-6 Induce Cell Migration in Differentiated HL-60 Cells

The instant example demonstrates that the compositions and methods of the invention are effective when practiced with a range of compounds of the invention. In particular, the instant example demonstrates that compounds 1-6, which were prepared according to one of the methods described in detail above, induce cell migration in differentiated HL-60 cells.

In vitro cell migration assays were done using differentiated HL-60 cells which are characterized as “neutrophil-like.” HL-60 cells were differentiated by adding 1.4% DMSO to RPMI-1640 media supplemented with 10% FBS and 100 μg/ml penicillin/streptomycin. Cells on day 6 following differentiation were used for in vitro migration assays.

Test compounds were diluted using migration media (RPMI-1640+0.1% BSA). Positive control chemoattractants and chemokines included 100 nM formyl-methionyl-leucyl-phenylalanine (fMLP), 30 ng/ml Stromal Cell-Derived Factor-1 (SDF-1), 300 ng/ml IL-8. Negative control was migration media alone.

In vitro migration assays were performed using a Neuroprobe migration apparatus (MBB96, Neuro Probe, Inc./16008 Industrial Drive, Gaithersburg, Md. 20877) according to the manufacturer's instructions with slight modifications. Briefly, ˜370 μl of diluted compound, chemokine or control was added to a 96-well fibronectin-coated, black-walled Packard plate (ensuring that there are no bubbles and that a slight positive meniscus forms on the top of the well). This plate was placed into the migration apparatus, an 8 μm framed filter was carefully placed on top of the Packard plate, and the apparatus was screwed down. The compound, chemokine or control was allowed to equilibrate at 37° C. for 15 minutes.

Differentiated HL-60 cells were then diluted to 4×10⁵ cells/ml in the migration media. 100 μl of cells were added to each of the top wells in the migration apparatus. Cells were allowed to migrate for 2 hours at 37° C.

After allowing migration, the apparatus was disassembled, and the Packard plate was centrifuged at 1500 rpm for 10 minutes in a tabletop centrifuge. Cells were allowed to adhere for 1 hour at 37° C.

Cells were then fixed for 20-30 minutes at ambient temperature by removing 180 μl of migration media and adding 180 μl of 8% paraformaldehyde.

After removing fixative solution, cells are washed with 200 μl phosphate-buffered saline for 5 minutes. Cells are stained by the addition of Hoechst stain diluted 1:1000 in phosphate-buffered saline and incubation at ambient temperature for 15 minutes.

After staining, the cells are washed with 200 μl phosphate-buffered saline (using a multichannel Rainin LTS pipetman to minimize disruption). Cell number in each well is quantitated using ImageXpress (Axon Instruments) or a fluorescent plate reader.

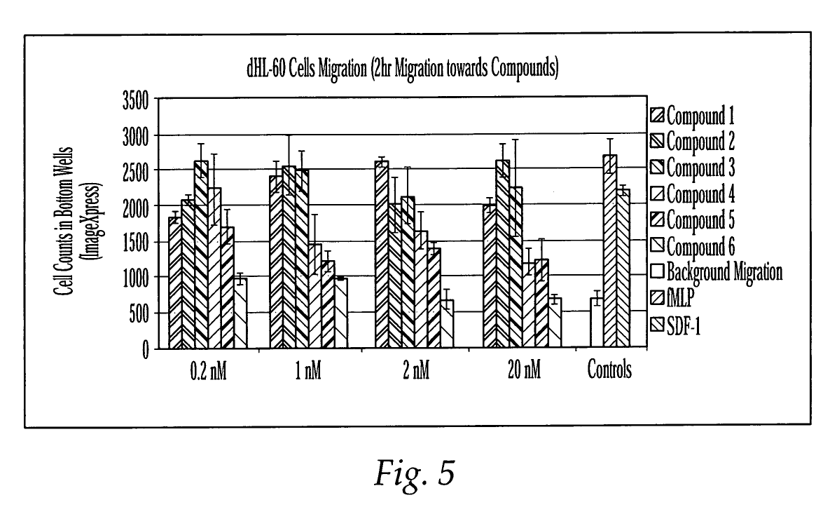

As shown in FIG. 5, compounds 1-6 induced significant migration of the cells in two hours.

### 6.7 Example 22

### Compound 17 Induces Cell Migration of RBL-2H3 Cells Expressing Various Chemokine Receptors

The instant example further demonstrates the effectiveness of the compositions and methods of the invention in modulating chemokine function. In particular, the instant example demonstrates that the methods and compositions of the invention are useful for the modulation of chemokine activity in cells expressing particular chemokine receptors.

RBL-2H3 cells were obtained from American Type Culture Collection (CRL-2256) and cultured in Minimum essential medium (MEM) with 10% heat-inactivated fetal bovine serum, 100 μg/ml penicillin/streptomycin. cDNA fragments encompassing chemokine receptor coding regions were obtained from cDNAs synthesized from HL-60 cells and/or human peripheral blood mononuclear cells by the polymerase chain reaction. Expression constructs of chemokine receptors were generated by cloning PCR fragments into expression vector pEF6/V5-TOPO (K9610-20, Invitrogen). Plasmid containing full-length human CXCR5 cDNA (human CXCR50TN00, pcDNA3.1+) was purchased from UMR cDNA Resource Center (Rolla, Mo.). Plasmid containing full-length human CCR7 cDNA (human CCR0700000, pcDNA3.1+) was purchased from UMR cDNA Resource Center (Rolla, Mo.). Transient transfections were carried out with Fugene 6 transfection reagent (1814443, Roche Applied Science) according to the manufacturer's instructions.

The RBL-chemokine receptor cells were used in migration assays 48 hours following transfection. In vitro migration assays were performed using a Neuroprobe 96-well chemotaxis chamber (MBA96, Neuro Probe, Inc./16008 Industrial Drive, Gaithersburg, Md. 20877) according to the manufacturer's instructions with slight modifications. Briefly, cells were trypsinized from culture flasks and allowed to recover in complete medium for 2 hours. Cells were then resuspended to 4×10⁵ cells/ml in migration medium (Ham's F12+0.1% BSA). A chemokines, compound 17 or control was added to the bottom wells of the migration chamber. 100 μl of cells were added to each of the top wells in the migration apparatus. Cells were allowed to migrate for 2 hours at 37° C.

Following the migration assays, cells that had migrated to the bottom wells were quantitated using CyQuant reagents according to the manufacturer's instructions (Molecular Probes, Eugene, Oreg.). Briefly, the residual cells in the top wells were removed by gentle aspiration and the bottom 96-well plate with filter intact was centrifuged at 1500 rpm for 10 minutes at 4° C., and the filter was removed and the media was emptied from the plate. The cells in the wells were lysed with Lysis/Cyquant/Detachment solution according to the manufacture's protocol. The fluorescence intensity (excitation wavelength at 485 nm and emission wavelength at 530 nm) was measured using Safire Spectrometer (Tecan; Research Triangle Park, N.C.). Compound 17 induced migration in RBL-2H3 cells that expressed chemokine receptors CXCR4 and CCR7, but not RBL-2H3 cells that expressed CXCR3 or CXCR5 cells.

### 6.8 Example 23

### Compound 1 is Effective In Vivo Mouse Model of Experimental Autoimmune Encephalomyelitis (EAE).

The instant example demonstrates that the methods and compositions of the invention can be used to treat or prevent a chemokine mediated condition such as multiple sclerosis in a subject. In particular, the instant example demonstrates that compound 1 is effective in vivo against a well known model of multiple sclerosis.

A mice experimental autoimmune encephalomyelitis (EAE) was used to test the efficacy of compound 1. EAE is a model for demyelinating disorders such as multiple sclerosis. See, e.g., Ransohoff et al., supra, at 576. The C57BL/6J mice model of EAE was used in the instant example. This is a chronic model of disease with animals developing an ascending paralysis that peaks in severity at approximately 16 days after immunization. After Day 16, severity decreases slightly and resolves to a stable phase.

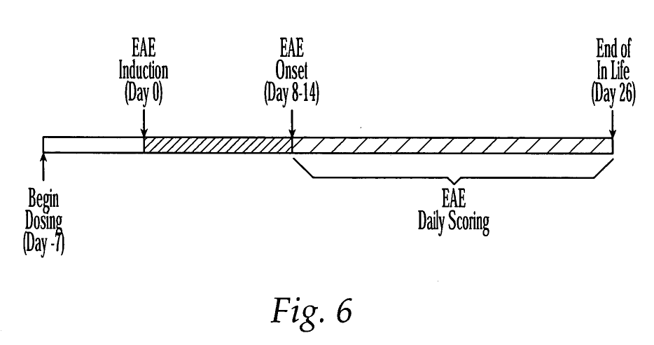

A shown in FIG. 6, compound 1 was tested in the C57BL/6J mice model of EAE disease prevention paradigm. 10 week old female C57BL/6J mice (Jackson Laboratory) were divided into six treatment groups: Vehicle only (0.5% HPMC, 0.2% SLS), Dexamethasone (1 mg/kg), and compound 1 at four doses: 0.3, 1, 3, 10 mg/kg. Animals were dosed once a day via oral gavage. Dosing of Compound 1 (0.3, 1, 3, 10 mg/kg), Vehicle, or Dexamethasone, was initiated 7 days prior to disease induction and continued until the end of the study. At Day 0, disease was induced. Scoring of clinical symptoms began on Day 9 post immunization, with the in-life phase completed on Day 26. On Day 26, animals were euthanized, and tissues and blood harvested for histopathology, pharmacokinetic analysis, and analysis of immunological function.

To induce EAE, animals were injected with myelin oligodendrocyte glycoprotein (MOG) emulsion subcutaneously (s.c.) in the left and right pectoral regions (50 ml each side). MOG (myelin oligodendrocyte glycoprotein (35-55) peptide (Sigma)) was dissolved in PBS to a final concentration of 2 mg/mL. Complete Freund's Adjuvant (CFA) was prepared by mixing 100 mg Mycobacterium tuberculosis with 25 mL IFA (4 mg/mL final concentration). To prepare the MOG antigen for immunization, the MOG peptide solution was mixed with an equal volume of CFA and the mixture loaded into glass syringes connected by a luer lock fitting. A stable emulsion was formed by passing the mixture back and forth through the fitting. After immunization, animals were given an intravenous injection of 400 ng Pertussis toxin (PT) dissolved in PBS. The PT treatment was repeated 2 days later.

Behavioral assessments of the animals were made daily during the active disease phase of the experiment (days 9-26 after disease induction). Clinical scores were assigned according to the scale described in Table 1. Scorers were blinded with respect to treatment status.

TABLE 1
EAE Scoring System for Mice

| EAE | |
|---|---|
| Score | Clinical Symptoms |
| 0 | Normal Animal-no overt signs of weakness |
| 1 | Limp tail |
| 2 | Hindlimb weakness-slipping on cagetop |
| 3 | Hindlimb paralysis-no weight bearing on |
| | hindlimbs |
| 4 | Forelimb paralysis-movement impaired |
| 5 | Moribund-euthanize for humane reasons |

At the end of the in-life phase of the study, ten animals each from the Vehicle and the Compound 1 (10 mg/kg) treatment groups were sacrificed for histological analysis. Animals were selected by ranking the entire treatment group by the average clinical score obtained during the last 6 days of the in-life phase, and then selecting every other animal for histological analysis.

Animals were euthanized by CO₂ inhalation and were transcardially perfused with 0.9% NaCl followed by 4% paraformaldehyde. After perfusion, brains and spinal columns were removed and post-fixed in 4% paraformaldehyde for 2 hours at 4°. Tissues were cryoprotected by overnight incubations in 10%, 20%, and 30% sucrose at 4°. Spinal cords were dissected out of the spinal column, and cut into 8 segments: C2-C5, C6-C8, T1-T3, T4-T6, T7-T9, T10-T12, L1-L3, L4-S1. These segments were embedded in OCT (Tissue Tek), and frozen at −20°. 20 μm sections were obtained from each spinal cord segment for histological analysis of cellular infiltration.

Analysis of cellular infiltrates was done on tissues stained with Hematoxylin and Eosin (H&E). Mounted sections were first dehydrated at 50° for 15 minutes. They were then rinsed in water for 2 minutes, and dehydrated through sequential changes of 70%, 95% and 100% ethanol. Following dehydration, slides were stained by the following treatments: 70% ethanol for 5 minutes, hematoxylin for 10 minutes, water for 2 minutes, acid alcohol for 30 seconds, blueing solution for 1 minute, water for 1 minute, eosin for 1 minute, several changes of water for 2 minutes. After staining, slides were dehydrated in the following sequence of solutions: 70% ethanol for 2 minutes, 95% ethanol for 2 minutes, 100% ethanol 2 times for 5 minutes, xylene 2 times for 5 minutes. Slides were then coverslipped using DPX mounting medium.

Tissue infiltration was evaluated using a qualitative scoring system. For each animal, one slide from each spinal cord level was scored for the presence and number of focal meningeal and/or perivascular infiltrates. The severity of these criteria was considered together to generate a tissue infiltration score as described in Table 2. The scores from all 8 levels were then averaged to obtain a mean infiltration score for each animal. In some cases, high quality sections were not available for a particular spinal cord level. In these instances, only scores from high quality sections were averaged to obtain a tissue infiltration score. Scorers were blinded with respect to treatment status.

TABLE 2
Scale for Scoring Tissue infiltration

| | # Focal meningeal | # Perivascular |
|---|---|---|
| Score | infiltrates | infiltrates |
| 0 | (—) | (—) |
| 1 | ≦3 | (—) |
| 2 | >3 | (—) |
| 3 | ≧5 | (—) |
| 4 | | 5-10 |
| 5 | | >10* |

Results were analyzed in the following ways: Daily average score was determined as the mean of all scores obtained in each treatment group each day. Maximum average score was calculated by averaging the highest clinical score obtained by each animal during the scoring period (Days 9-26). Cumulative score was calculated by summing all scores obtained during the scoring period (Day 9-26) for each animal. Individual cumulative scores were used to calculate the group average. For analysis of daily average, maximum average, and cumulative average scores, the Compound 1 (0.3, 1, 3, 10 mg/kg) and Vehicle groups were compared using Kruskal-Wallis ANOVA by ranks. Pairwise comparisons between Compound 1 treatment groups and the Vehicle group were done using the Mann-Whitney U test.

Average body weights were analyzed with one way ANOVA. Pairwise comparisons between average weights of the Compound 1 (10 mg/kg) group and the Compound 1 (0.3, 1 mg/kg) groups were done using the Student's T test.

For comparison of tissue infiltration between the Vehicle treated and Compound 1 treated animals, the Student's T test was used.

For statistical comparisons, all animals in each treatment group were included with the exception of animal B390, which displayed hindlimb weakness prior to disease induction, and animal B447, which was euthanized on Day 3 due to a gavage-induced injury.

All statistical analysis was done using Statistica (Release 6) or GraphPad Prism.

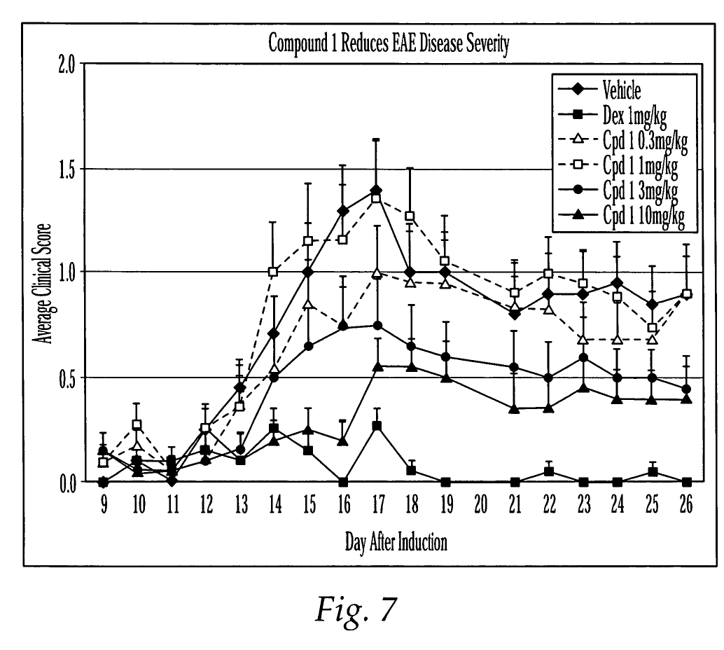

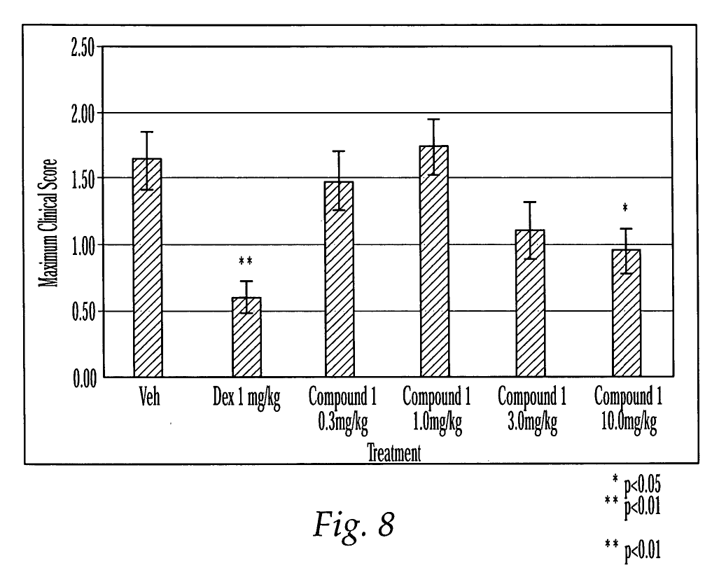

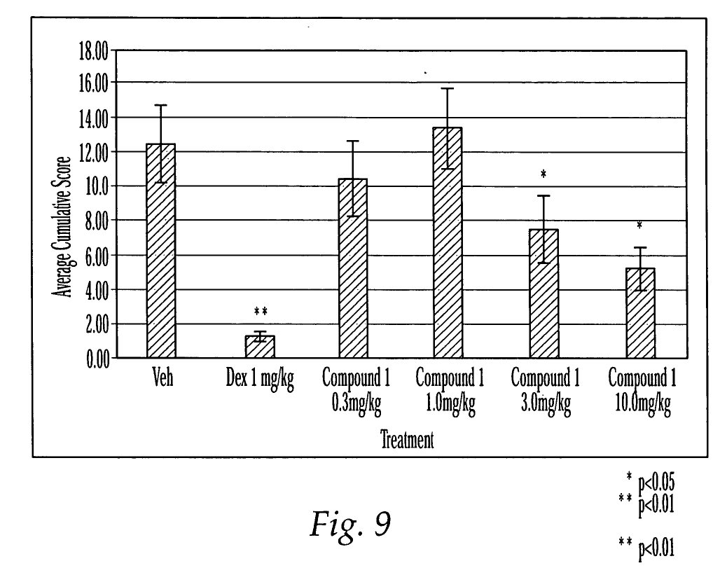

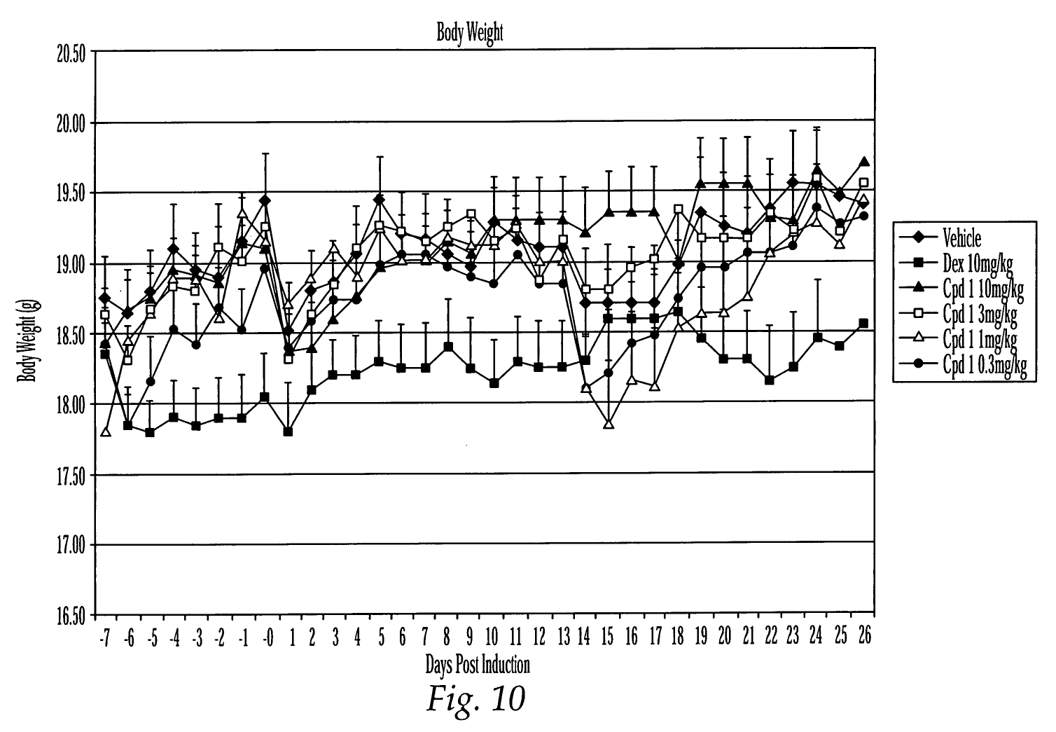

As shown in FIG. 7 to FIG. 11, administration of Compound 1 was effective in preventing and treatment of EAE in the mice model. Particularly, FIG. 7 shows that dosing of Compound 1 caused a significant reduction in disease severity by reduding average clinical scores. In addition, administration of Compound 1 also had a significant effect on other measures of disease severity including maximum disease score (see FIG. 8) and cumulative disease score (see FIG. 9). Moreover, FIG. 10 shows that high dose of Compound 1 (10 mg/kg) had a significant effect on reversal of EAE-associated weight loss.

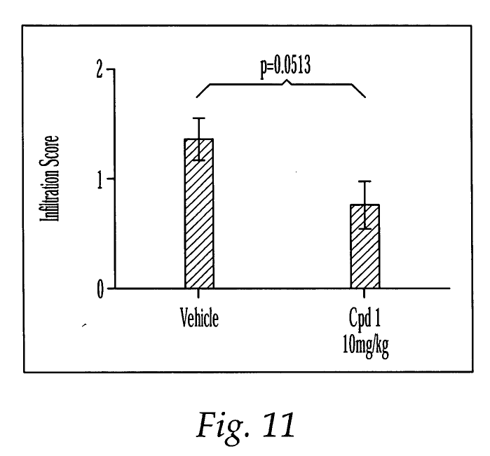

Cellular infiltration is commonly seen in EAE animals. As shown in FIG. 11, mice treated with Compound 1 had an lower infiltration score than control mice, i.e., mice treated with Vehicle. While failing to reach statistical significance (p=0.0513), these data strongly suggest that Compound 1 treatment can reduce the inflammatory infiltration in a manner that is entirely consistent with its effects on leukocyte migration in vitro.

As demonstrated in this example, the methods and compositions of the invention are effective in vivo for the prevention and treatment of multiple sclerosis in a well know animal model system.

### 6.9 Example 23

### α-(4-methoxymethoxy-3,5-di-tert-butyl-phenyl)-(N-tert-butyl)-nitrone (Compound 15) is Effective In Vivo Against a Rat Model Demyelinating Disorder

The instant example demonstrates that the methods and compositions of the invention can be used to treat or prevent a chemokine mediated condition such as multiple sclerosis in a subject. In particular, the instant example demonstrates that compound 1 is effective in vivo against a well known model of multiple sclerosis.

A rodent experimental autoimmune encephalomyelitis (EAE) was used to test the efficacy of compound 1. EAE is a model for demyelinating disorders such as multiple sclerosis. See, e.g., Ransohoffet al., supra, at 576.

In this exemplary study, α-(4-methoxymethoxy-3,5-di-tert-butyl-phenyl)-(N-tert-butyl)-nitrone (compound 15) was administered according to the methods of the invention. It is believed that compound 15 can be converted into compound 1 by a subject after administration to the subject; i.e., compound 15 is a prodrug of compound 1 as described in the methods of the invention, above.

7- to 10-week female Lewis rats (The Jackson Laboratory, Bar Harbor, Me.) were used since they are susceptible to EAE, and since MS occurs at a 2:1 ratio between females and males respectively. Rats were immunized either at the base of the tail (or in one hind footpad) with myelin antigen myelin basic peptide, MBP 85-99. Peptide antigens were mixed with complete Freund's adjuvant including killed mycobacterium tuberculosis at a doses of 4 mg/ml. 200 micrograms of antigen mixed with the 0.1 ml emulsion of adjuvant were used. The myelin antigens were dissolved in phosphate buffered saline. Control rats did not receive myelin antigens in the mixture.

Nine rats were used in each of the following groups: EAE control, vehicle control, compound 15 (prevention), compound 15 (pro-treatment), phenyl-N-butyl-nitrone (prevention) and phenyl-N-butyl-nitrone (pro-treatment). For the treatment model, fifteen rats were used in each of three groups: EAE control, vehicle control, compound 15 (treatment). Drugs were administered via oral gavage feeding. Rats were dosed daily after the first dose until recovery from acute disease (typically 20 days after EAE induction). Compound 15 was dosed at 10 mg/kg/day and phenyl-N-butyl-nitrone was dosed at 100 mg/kg/day. In the prevention model, first dose of the drugs was administered 7 days prior to EAE induction. In the pro-treatment model, first dose was administered 4 days after EAE induction. In the treatment model, first dose was administered at the peak of the disease after animals were randomized into equal groups.

All rats were followed daily for clinical signs and weighed every four days. Longer water tubes were used to allow mice to drink when they develop paralysis, and chow was put on the floor of the cage to facilitate nourishment if the rats were paralyzed. Rats were scored as follows: 0, healthy; 1, tail weakness or paralysis; 2, paraparesis (incomplete paralysis of one or two hind limbs or plegia of one hind limb); 3, paraplegia extending to the thorax; 4, forelimb weakness, paralysis with hind limb paraparesis or paraplegia; 5, moribund or dead animal.

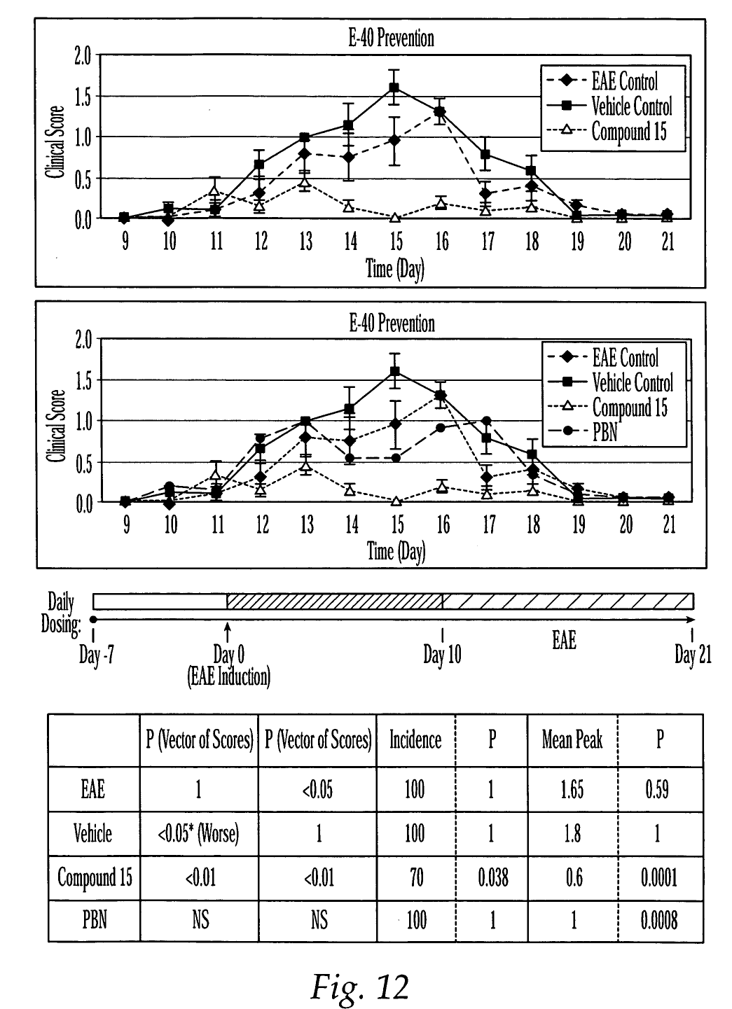

As shown in FIG. 12, administration of compound 15 was effective in preventing EAE in the rat model system. In particular, administration of compound 15 yielded significantly better clinical scores when compared to controls and when compared to phenyl-N-butyl-nitrone (“PBN”), a known nitrone compound.

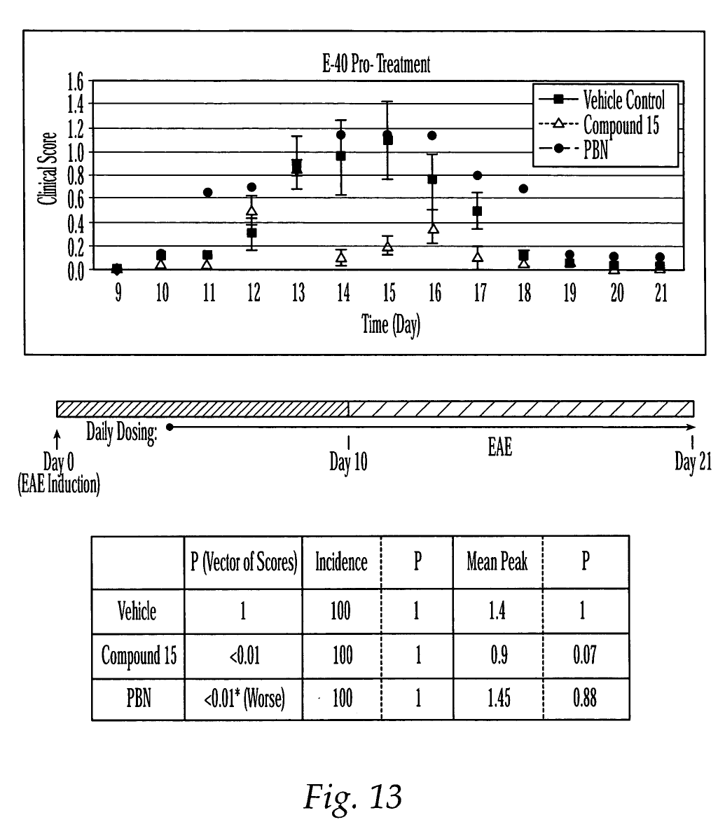

As shown in FIG. 13, administration of compound 15 was effective in the pro-treatment of the rat EAE model. Dosing of compound 15 four days after induction of EAE yielded significantly better clinical scores when compared to control or to PBN. Significantly, the clinical scores of rats treated with compound 15 improved dramatically on day 15, ten days after the rats were initially treated with the compound.

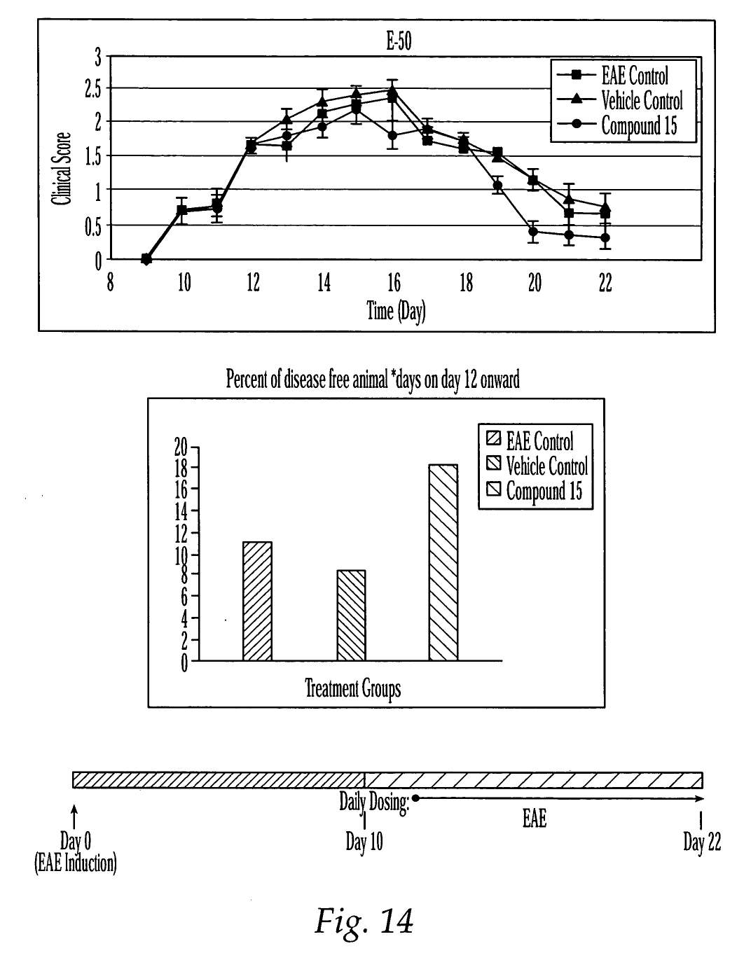

Finally, FIG. 14 shows that administration of compound 15 was effective in the treatment of EAE. Rats were treated with compound 15 at the peak of EAE, and significantly more rats treated with compound 15 were disease free following administration when compared to controls.

As demonstrated in this example, the methods and compositions of the invention are effective in vivo for the prevention and treatment of chemokine mediated disorders such as multiple sclerosis in a well known animal model system.

From the foregoing description, various modifications and changes in the compositions and methods of this invention will occur to those skilled in the art. All such modifications coming within the scope of the appended claims are intended to be included therein. All references cited herein are hereby incorporated by reference in their entireties.

1. A pharmaceutical composition comprising: an effective multiple sclerosis-treating amount of an aryl nitrone in a pharmaceutically acceptable carrier, excipient or diluent, wherein the aryl nitrone is according to formula I: 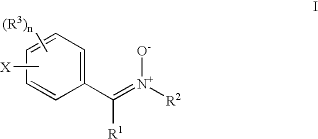
wherein
n is an integer from 1 to 4;
X is —OH or a salt thereof;
R¹ is selected from the group consisting of hydrogen, alkyl, cycloalkyl and aryl;
R² is selected from the group consisting of alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heterocycloalkyl, substituted heterocycloalkyl, heterocycloalkenyl, substituted heterocycloalkenyl, heteroaryl, substituted heteroaryl, benzyl and substituted benzyl;
each R³ is independently selected from the group consisting of aryl, heteroaryl and the following formula: 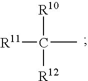
R¹⁰ is selected from the group consisting of hydrogen, lower alkyl and lower cycloalkyl; or R¹⁰ and R¹¹ can be joined to form an alkylene, substituted alkylene, or heteroalkylene group;
and R¹¹ and R¹² are independently selected from the group consisting of hydrogen, lower alkyl and lower cycloalkyl; or R¹¹ and R¹² can be joined to form an alkylene group having from 2 to 10 carbon atoms;
or a prodrug, pharmaceutically-acceptable salt or solvate thereof.

2. The pharmaceutical composition of claim 1 wherein the aryl nitrone is according to formula Ia: 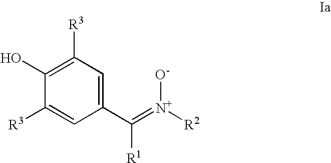
or a prodrug, pharmaceutically-acceptable salt or solvate thereof.

3. The composition of claim 1 wherein R¹ is hydrogen or lower alkyl.

4. The composition of claim 1 wherein R¹ is hydrogen or alkyl having 1 to 4 carbon atoms, more preferably 1 or 2 carbon atoms.

5. The composition of claim 1 wherein R¹ is hydrogen.

6. The composition of claim 1 wherein R² is selected from the group consisting of alkyl, substituted alkyl and cycloalkyl.

7. The composition of claim 1 wherein R² is alkyl having 3 to 6 carbon atoms or cycloalkyl having 5 to 6 carbon atoms.

8. The composition of claim 1 wherein R² is selected from the group consisting of methyl, n-propyl, isopropyl, 1-hydroxy-2-methylprop-2-yl, n-butyl, and tert-butyl and cyclohexyl.

9. The composition of claim 1 wherein R¹⁰, R¹¹ and R¹² are each independently lower alkyl.

10. The composition of claim 1 wherein R¹⁰, R¹¹ and R¹² are each methyl.

11. The composition of claim 1 wherein each R³ is independently lower alkyl.

12. The composition of claim 1 wherein each R³ independently has the formula:

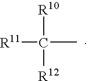

13. The composition of claim 1 wherein each R³ is selected from the group consisting of methyl, ethyl, butyl, propyl and cyclohexyl.

14. The composition of claim 1 wherein each R³ is selected from the group consisting of methyl, isopropyl and tert-butyl.

15. The composition of claim 1 wherein each R³ is cyclohexyl.

16. The composition of claim 1 wherein each R³ is methyl.

17. The composition of claim 1 wherein each R³ is isopropyl.

18. The composition of claim 1 wherein one R³ is methyl and the other R³ is tert-butyl.

19. The composition of claim 1 wherein the aryl nitrone is selected from the group consisting of α-(4-hydroxy-3,5-di-tert-butylphenyl)-N-tert-butylnitrone; α-(4-hydroxy-3,5-di-phenylphenyl)-N-tert-butylnitrone; α-(4-hydroxy-3-tert-butylphenyl)-N-tert-butylnitrone; α-(6-hydroxy-3,5-di-tert-butylphenyl)-N-tert-butylnitrone; and α-(6-hydroxy-5-methylphenyl)-N-tert-butylnitrone or any combination thereof.

20. A unit dosage form of the composition of claim 1 comprising about 1 to 1750 mg of the aryl nitrone.

21. A unit dosage form of the composition of claim 1 comprising about 1 to 1500 mg of the aryl nitrone.

22. A unit dosage form of the composition of claim 1 comprising about 1 to 1250 mg of the aryl nitrone.

23. A unit dosage form of the composition of claim 1 comprising about 1 to 1000 mg of the aryl nitrone.

24. A unit dosage form of the composition of claim 1 comprising about 1 to 750 mg of the aryl nitrone.

25. A unit dosage form of the composition of claim 1 comprising about 250 to 750 mg of the aryl nitrone.

26. A unit dosage form of the composition of claim 1 comprising about 500 to 750 mg of the aryl nitrone.

27. A pharmaceutical composition comprising: an effective multiple sclerosis-treating amount of an aryl nitrone in a pharmaceutically acceptable carrier, excipient or diluent, wherein the aryl nitrone is according to formula II: 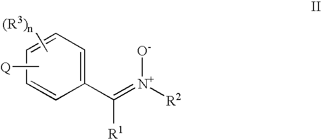
wherein
n is an integer from 1 to 4;
Q is —OR;
R is selected from the group consisting of: 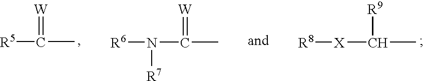
X is oxygen, sulfur, —S(O)— or —S(O)₂—; and
W is oxygen or sulfur;
R¹ is selected from the group consisting of hydrogen, alkyl, cycloalkyl and aryl;
R² is selected from the group consisting of alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heterocycloalkyl, substituted heterocycloalkyl, heterocycloalkenyl, substituted heterocycloalkenyl, heteroaryl, substituted heteroaryl, benzyl and substituted benzyl;
each R³ is independently selected from the group consisting of aryl, heteroaryl and the following formula: 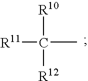
R⁵ is selected from the group consisting of alkyl, substituted alkyl, alkenyl, substituted alkenyl, alkynyl, substituted alkynyl, cycloalkyl, substituted cycloalkyl, cycloalkenyl and substituted cycloalkenyl;
R⁶ and R⁷ are independently selected from the group consisting of hydrogen, alkyl, substituted alkyl, alkenyl, substituted alkenyl, alkynyl, substituted alkynyl, cycloalkyl, substituted cycloalkyl, cycloalkenyl and substituted cycloalkenyl; or R^{6 and R}⁷ can be joined to form an alkylene or substituted alkylene group having from 2 to 10 carbon atoms;
R⁸ is selected from the group consisting of alkyl, substituted alkyl, alkenyl, substituted alkenyl, alkynyl, substituted alkynyl, cycloalkyl, substituted cycloalkyl, cycloalkenyl and substituted cycloalkenyl;
R⁹ is selected from the group consisting of hydrogen, alkyl, substituted alkyl, alkenyl, substituted alkenyl, alkynyl, substituted alkynyl, cycloalkyl, substituted cycloalkyl, cycloalkenyl and substituted cycloalkenyl; or R⁸ and R⁹ can be joined to form an alkylene or substituted alkylene group having from 2 to 10 carbon atoms;
R¹⁰ is selected from the group consisting of hydrogen, lower alkyl and lower cycloalkyl; or R¹⁰ and R¹¹ can be joined to form an alkylene, substituted alkylene or heteroalkylene group;
R¹¹ and R¹² are independently selected from the group consisting of hydrogen, lower alkyl and lower cycloalkyl; or R¹¹ and R¹² can be joined to form an alkylene group having from 2 to 10 carbon atoms; and
zero, one, two or three of the carbon atoms of the phenyl ring in formula I are substituted with a heteroatom;
or a prodrug or pharmaceutically acceptable salt or solvate thereof.

28. The pharmaceutical composition of claim 27 wherein the aryl nitrone is according to formula IIa: 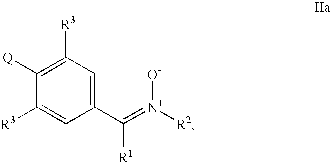
or a prodrug, pharmaceutically-acceptable salt or solvate thereof.

29. The composition of claim 27 wherein W is oxygen.

30. The composition of claim 28 wherein R¹ is hydrogen or lower alkyl.

31. The composition of claim 29 wherein R¹ is hydrogen.

32. The composition of claim 27 wherein R² is selected from the group consisting of alkyl, substituted alkyl and cycloalkyl.

33. The composition of claim 27 wherein R² is selected from the group consisting of methyl, n-propyl, isopropyl, 1-hydroxy-2-methylprop-2-yl, n-butyl, tert-butyl, 3-thiomethylpropyl, 3-(thiomethoxy)but-1-yl, cyclohexyl, 4-trifluoromethybenzyl and 3,4,5-trimethoxybenzyl.

34. The composition of claim 27 wherein R⁵ is selected from the group consisting of alkyl and cycloalkyl.

35. The composition of claim 34 wherein R⁵ is selected from the group consisting of methyl, ethyl, n-propyl, isopropyl and n-butyl.

36. The composition of claim 27 wherein R⁷ is hydrogen and R⁶ is selected from the group consisting of alkyl and alkoxycarbonylalkyl.

37. The composition of claim 36 wherein R⁶ groups is selected from the group consisting of ethyl, n-propyl, isopropyl, n-butyl, ethoxycarbonylmethyl and 2-(ethoxycarbonyl)ethyl.

38. The composition of claim 27 wherein X is oxygen; R⁹ is hydrogen; and R⁸ is alkyl or alkoxyalkyl.

39. The composition of claim 38 wherein R⁸ is selected from the group consisting of methyl and methoxyethyl.

40. The composition of claim 27 wherein R¹⁰, R¹¹ and R¹² are independently lower alkyl.

41. The composition of claim 40 wherein R¹⁰, R¹¹ and R¹² are methyl.

42. The composition of claim 27 wherein R¹ is hydrogen or lower alkyl.

43. The composition of claim 27 wherein R¹ is hydrogen or alkyl having 1 to 4 carbon atoms, more preferably 1 or 2 carbon atoms.

44. The composition of claim 27 wherein each R³ independently has the formula:

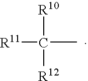

45. The composition of claim 27 wherein each R³ is selected from the group consisting of methyl, ethyl, butyl, propyl and cyclohexyl.

46. The composition of claim 27 wherein each R³ is selected from the group consisting of methyl, isopropyl and tert-butyl.

47. The composition of claim 27 wherein each R³ is cyclohexyl.

48. The composition of claim 27 wherein each R³ is methyl.

49. The composition of claim 27 wherein each R³ is isopropyl.

50. The composition of claim 27 wherein one R³ is methyl and the other R³ is tert-butyl.

51. The composition of claim 27 wherein the aryl nitrone is α-(4-methoxymethoxy-3,5-di-tert-butyl-phenyl)-(N-tert-butyl)-nitrone.

52. A method for treating a subject with multiple sclerosis or a related condition, which method comprises administering to said subject a pharmaceutical composition according to claim 1, 2, 27 or 28.

53. A method for treating a subject with multiple sclerosis or a related condition, which method comprises administering to said subject a therapeutically effective amount of a 3,4,5 tri-substituted aryl nitrone.

54. The method of claim 53 wherein the subject is a mammal.

55. The method of claim 53 wherein the subject is a human.

56. The method of claim 53 wherein the aryl nitrone is according to formula I: 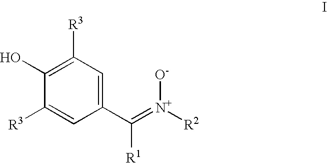
wherein
R¹ is selected from the group consisting of hydrogen, alkyl, cycloalkyl and aryl;
R² is selected from the group consisting of alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heterocycloalkyl, substituted heterocycloalkyl, heterocycloalkenyl, substituted heterocycloalkenyl, heteroaryl, substituted heteroaryl, benzyl and substituted benzyl;
each R³ is independently selected from the group consisting of aryl, heteroaryl and the following formula: 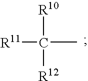
R¹⁰ is selected from the group consisting of hydrogen, lower alkyl and lower cycloalkyl; or R¹⁰ and R¹¹ can be joined to form an alkylene, substituted alkylene, or heteroalkylene group;
and R¹¹ and R¹² are independently selected from the group consisting of hydrogen, lower alkyl and lower cycloalkyl; or R¹¹ and R¹² can be joined to form an alkylene group having from 2 to 10 carbon atoms;
or a prodrug or pharmaceutically acceptable salt or solvate thereof.

57. The method of claim 53 wherein the aryl nitrone is selected from the group consisting of α-(4-hydroxy-3,5-di-tert-butylphenyl)-N-tert-butylnitrone, α-(4-hydroxy-3,5-di-phenylphenyl)-N-tert-butylnitrone, α-(4-hydroxy-3-tert-butylphenyl)-N-tert-butylnitrone, α-(6-hydroxy-3,5-di-tert-butylphenyl)-N-tert-butylnitrone, and α-(6-hydroxy-5-methylphenyl)-N-tert-butylnitrone.

58. The method of claim 53 wherein the aryl nitrone is according to formula II: 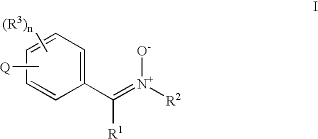
wherein
n is an integer from 1 to 4;
Q is —OR;
R is selected from the group consisting of: 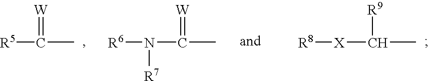
X is oxygen, sulfur, —S(O)— or —S(O)₂—; and
W is oxygen or sulfur;
R¹ is selected from the group consisting of hydrogen, alkyl, cycloalkyl and aryl;
R² is selected from the group consisting of alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heterocycloalkyl, substituted heterocycloalkyl, heterocycloalkenyl, substituted heterocycloalkenyl, heteroaryl, substituted heteroaryl, benzyl and substituted benzyl;
each R³ is independently selected from the group consisting of aryl, heteroaryl and the following formula: 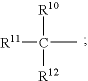
R⁵ is selected from the group consisting of alkyl, substituted alkyl, alkenyl, substituted alkenyl, alkynyl, substituted alkynyl, cycloalkyl, substituted cycloalkyl, cycloalkenyl and substituted cycloalkenyl;
R⁶ and R⁷ are independently selected from the group consisting of hydrogen, alkyl, substituted alkyl, alkenyl, substituted alkenyl, alkynyl, substituted alkynyl, cycloalkyl, substituted cycloalkyl, cycloalkenyl and substituted cycloalkenyl; or R⁶ and R⁷ can be joined to form an alkylene or substituted alkylene group having from 2 to 10 carbon atoms;
R⁸ is selected from the group consisting of alkyl, substituted alkyl, alkenyl, substituted alkenyl, alkynyl, substituted alkynyl, cycloalkyl, substituted cycloalkyl, cycloalkenyl and substituted cycloalkenyl;
R⁹ is selected from the group consisting of hydrogen, alkyl, substituted alkyl, alkenyl, substituted alkenyl, alkynyl, substituted alkynyl, cycloalkyl, substituted cycloalkyl, cycloalkenyl and substituted cycloalkenyl; or R⁸ and R⁹ can be joined to form an alkylene or substituted alkylene group having from 2 to 10 carbon atoms;
R¹⁰ is selected from the group consisting of hydrogen, lower alkyl and lower cycloalkyl; or R¹⁰ and R¹¹ can be joined to form an alkylene, substituted alkylene or heteroalkylene group;
R¹¹ and R¹² are independently selected from the group consisting of hydrogen, lower alkyl and lower cycloalkyl; or R¹¹ and R¹² can be joined to form an alkylene group having from 2 to 10 carbon atoms; and
zero, one, two or three of the carbon atoms of the phenyl ring in formula I are substituted with a heteroatom;
or a prodrug, pharmaceutically-acceptable salt or solvate thereof.

59. The method of claim 53 wherein the aryl nitrone is α-(4-methoxymethoxy-3,5-di-tert-butyl-phenyl)-N-tert-butylnitrone.

60. A method of treatment or prevention comprising: administering to a patient in need thereof an effective multiple sclerosis-treating or preventing amount of a pharmaceutical composition according to claim 1, 20, or 27.

61. A method of treatment comprising: administering to a patient in need thereof an amount of a pharmaceutical composition according to claim 1, 20, or 27 effective to treat multiple sclerosis.

62. A method of prophylaxis comprising: administering to a patient in need thereof an amount of a pharmaceutical composition according to claim 1, 20, or 27 effective to prevent multiple sclerosis.

63. A method of modulating chemokine function in a subject comprising administering to the subject a pharmaceutical composition according to claim 1 or 20.

64. A method of modulating chemokine function in a cell comprising contacting the cell with an aryl nitrone.

65. The method of claim 64 wherein the aryl nitrone is a 3,4,5 tri-substituted aryl nitrone.

66. The method of claim 65 wherein the aryl nitrone is according to formula I: 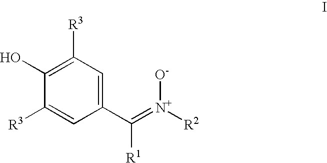
wherein
R¹ is selected from the group consisting of hydrogen, alkyl, cycloalkyl and aryl;
R² is selected from the group consisting of alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heterocycloalkyl, substituted heterocycloalkyl, heterocycloalkenyl, substituted heterocycloalkenyl, heteroaryl, substituted heteroaryl, benzyl and substituted benzyl;
each R³ is independently selected from the group consisting of aryl, heteroaryl and the following formula: 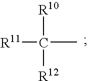
R¹⁰ is selected from the group consisting of hydrogen, lower alkyl and lower cycloalkyl; or R¹⁰ and R¹¹ can be joined to form an alkylene, substituted alkylene, or heteroalkylene group;
and R¹¹ and R¹² are independently selected from the group consisting of hydrogen, lower alkyl and lower cycloalkyl; or R¹¹ and R¹² can be joined to form an alkylene group having from 2 to 10 carbon atoms;
or a prodrug, pharmaceutically-acceptable salt or solvate thereof.

67. The method of claim 65 wherein the aryl nitrone is selected from the group consisting of α-(4-hydroxy-3,5-di-tert-butylphenyl)-N-tert-butylnitrone, α-(4-hydroxy-3,5-di-phenylphenyl)-N-tert-butylnitrone, α-(4-hydroxy-3-tert-butylphenyl)-N-tert-butylnitrone, α-(6-hydroxy-3,5-di-tert-butylphenyl)-N-tert-butylnitrone, and α-(6-hydroxy-5-methylphenyl)-N-tert-butylnitrone.
